(19) United States
(12) Patent Application Publication
(10) Pub. No.: US 2021/0024715 A1
(43) Pub. Date: Jan. 28, 2021

(54) SPHERICAL MICROPARTICLES

(71) Applicant: BASF SE, Ludwigshafen am Rhein (DE)

(72) Inventors: Bernd Dieter OSCHMANN, Ludwigshafen am Rhein (DE); Wolfgang KRAUSE, Lampertheim (DE); Patrick LEIBACH, Ludwigshafen am Rhein (DE); Kerstin MUELHEIMS, Ludwigshafen am Rhein (DE); Ralf PELZER, Lampertheim (DE); Ewelina BURAKOWSKA-MEISE, Ludwigshafen am Rhein (DE)

(21) Appl. No.: 17/045,691

(22) PCT Filed: Apr. 4, 2019
(86) PCT No.: PCT/EP2019/058500

§ 371 Date: Oct. 6, 2020

(30) Foreign Application Priority Data
Apr. 6, 2018 (EP) 18166159.6

(51) Int. Cl. C08J 9/00; A61K 8/11; A61K 8/02; A61K 8/85; A61K 9/16; C11D 3/50; C11D 3/37

(52) U.S. Cl. CPC C08J 9/0066; A61K 8/11; A61K 8/025; A61K 8/85; C08J 2467/04; C11D 3/505; C11D 3/3715; A61K 2800/10; C08J 2367/02; A61K 9/1647

(57) ABSTRACT

The present invention relates to a composition of spherical microparticles composed of a wall material and at least one cavity that comprises a gas and/or a liquid, which have pores on the surface thereof, wherein the spherical microparticles have a mean particle diameter of 10-600 μm and wherein at least 80% of those microparticles, the particle diameter of which does not deviate from the mean particle diameter of the microparticles of the composition by more than 20%, each have on average at least 10 pores, the diameter of which is in the range from 1/5000 to 1/5 of the mean particle diameter, and, furthermore, the diameter of each of these pores is at least 20 nm, wherein the wall material consists of a composition comprising at least one aliphatic-aromatic polyester and at least one additional polymer, wherein the additional polymer is selected from the group consisting of polyhydroxy fatty acids, poly(p-dioxanones), polyanhydrides, polyesteramides, polysaccharides and proteins, to a method for the preparation thereof and use thereof.

The present invention relates to a method for preparing spherical microparticles, to the fillable spherical microparticles obtainable by this method, and also to the use thereof.

Microcapsules, like porous microparticles, are used as carrier for active substances, which as a result can be better processed, formulated, or released in a controlled manner.

Thus, in the medical sector, microparticles based on biopolymers for the controlled release of active compounds are known. In “Acta Biomaterialia” 10(2914) 5090-5098, porous microspheres having a scaffold made of a copolymer (PLGA) of lactic acid and hydroxyacetic acid (glycolic acid) and having a mean particle diameter of 84 μm are described.

Jian-Qing Hu et al, “Journal of Central South University of Technology”, vol. 18, No. 2, (2011-04-01), pages 337-342, describes the preparation of microcapsules comprising a polyfunctional aziridine as capsule core. Such microcapsules are tight, and are intended to release the crosslinker as required, by destruction of the capsule wall. The capsules are formed from a w/o/w emulsion, by the oil phase comprising a polyester dissolved in dichloromethane, and the wall being formed by removal of the solvent. The wall material is a polyester made of dimethylphthalate, glycol and 1,3-propanediol.

DE 3428640 teaches the production of microporous pulverulent polylactides and the use thereof for the controlled administration of active compounds.

Furthermore, WO 2015/070172 teaches porous microspheres made of PLGA, the pores of which have been loaded with proteins and the pores of which are closed by heating. The addition of magnesium carbonate or zinc carbonate to modify the pH brings about an improvement in the intake of the proteins.

Furthermore, US 2005/0069591 teaches porous microspheres made of a biodegradable polymer such as PLGA, which are prepared via a double water/oil/water emulsion. The microspheres are subsequently loaded with proteins.

EP 467 528 teaches polymeric carrier particles having particle sizes up to 250 μm and pores at the surface thereof, wherein the maximum pore size is 0.4 μm. The material of the carrier particles is in this case prepared by polymerization of styrene and a polyester of maleic anhydride/phthalic anhydride/propylene glycol. The polyester serves as crosslinker in this radical polymerization. The radical polymerization in this case is carried out as bulk polymerization, with the polyester being directly polymerized in the styrene.

The microporous polymers of the prior art are customarily loaded with medical active compounds or proteins and are intended to administer these in a controlled manner in the form of medicaments. Longer storage is not required in this case. Furthermore, such substances are hydrophilic.

If it is desired to provide aroma chemicals in a form that is readily handled, e.g. in the form of microparticles, other requirements must be met. Such microparticles should have good long-term stability, that is to say a good shelf life. For this, the microparticles themselves must be stable to the aroma chemicals, which of course are generally hydrophobic.

It was therefore an object of the present invention to provide microparticles which can be readily filled with an aroma chemical and subsequently closed. The resulting present aroma chemical preparations should have a good shelf life. Of particular interest are microparticles which can be filled with at least one aroma chemical and which release these aroma chemical(s) only after a period of latency. It is of further interest that the aroma profile of the aroma chemical(s) is retained during the release. Advantageously, the microparticles should have good biodegradability, be simple to prepare and be suitable for a broad spectrum of applications.

Accordingly, a method for preparing spherical microparticles was found, in which
a) an emulsion is prepared from an aqueous solution of a pore former as discontinuous phase and a continuous phase comprising a solution of at least one aliphatic-aromatic polyester and at least one additional polymer in which the additional polymer is selected from the group consisting of polyhydroxy fatty acids, poly(p-dioxanones), polyanhydrides, polyesteramides, polysaccharides and proteins, in a water-immiscible solvent,
b) the w/o emulsion obtained in a) is emulsified in water in the presence of at least one dispersant to give a w/o/w emulsion having droplets with a mean size of 1-600 μm, and the water-immiscible solvent is removed at a temperature in the range from 20 to 80° C.,
c) the spherical microparticles formed in method step b) are separated off and optionally dried.

Furthermore, the spherical microparticles obtainable by this method, the use thereof as carrier for aroma chemicals, a method for the filling thereof with at least one aroma chemical and the filled spherical microparticles obtained thereby, were also found.

Furthermore, the use of the optionally closed microparticles filled with at least one aroma chemical in perfumes, washing and cleaning agents, cosmetic agents, body care agents, hygiene articles, aroma compositions, food, food supplements, scent dispensers and fragrances was found, and also the use thereof for the controlled release of aroma chemicals.

Furthermore, compositions of spherical microparticles composed of a wall material and at least one cavity that comprises a gas and/or a liquid were found, which have pores on the surface thereof, wherein the spherical microparticles have a mean particle diameter of 10-600 μm, wherein the spherical microparticles have a mean particle diameter of 10-600 μm and wherein at least 80% of those microparticles, the particle diameter of which does not deviate from the mean particle diameter of the microparticles of the composition by more than 20%, each have on average at least 10 pores, the diameter of which is in the range from 1/5000 to 1/5 of the mean particle diameter, and, furthermore, the diameter of each of these pores is at least 20 nm, in which the wall material consists of a composition comprising at least one aliphatic-aromatic polyester and at least one additional polymer, in which the additional polymer is selected from the group consisting of polyhydroxy fatty acids, poly(p-dioxanones), polyanhydrides, polyesteramides, polysaccharides and proteins and the wall material has a solubility in dichloromethane at 25° C. of at least 50 g/1.

The statement regarding the state of matter of the substance contained in the cavity of the microparticle relates to 20° C. (room temperature) and 1 bar.

The present invention therefore relates to a composition of spherical microparticles composed of a wall material and at least one cavity that comprises a gas and/or a liquid, which have pores on the surface thereof, wherein the spherical microparticles have a mean particle diameter of 10-600 μm and wherein at least 80% of those microparticles, the particle diameter of which does not deviate from the mean particle diameter of the microparticles of the composition by more than 20%, each have on average at least 10 pores, the diameter of which is in the range from 1/5000 to 1/5 of the mean particle diameter, and, furthermore, the diameter of each of these pores is at least 20 nm,

in which the wall material consists of a composition comprising at least one aliphatic-aromatic polyester and at least one additional polymer, in which the additional polymer is selected from the group consisting of polyhydroxy fatty acids, poly(p-dioxanones), polyanhydrides, polyesteramides, polysaccharides and proteins.

The invention is associated with a number of advantages:
the microparticles are producible in a simple and inexpensive manner.
the filling of the microparticles is possible in various ways
whether and to what extent the pores of the filled microparticles are sealed can be freely selected
sealing of the pores is possible even with only low thermal stress of the filled microparticles
the release characteristics of the aroma substance can be specifically controlled by the choice of wall material and the type of filling.
the microparticles laden with the aroma chemical can be stored over a prolonged period without any significant loss of aroma chemical
the aroma profile is retained during the release of the aroma chemical or the mixture of aroma chemicals.
by selection of the wall material, the microparticles can be configured such that they are biodegradable.

Furthermore, the following embodiments were found:
1. A composition of spherical microparticles composed of a wall material and at least one cavity that comprises a gas and/or a liquid, which have pores on the surface thereof, wherein the spherical microparticles have a mean particle diameter of 10-600 μm and wherein at least 80% of those microparticles, the particle diameter of which does not deviate from the mean particle diameter of the microparticles of the composition by more than 20%, each have on average at least 10 pores, the diameter of which is in the range from 1/5000 to 1/5 of the mean particle diameter, and, furthermore, the diameter of each of these pores is at least 20 nm,
   in which the wall material consists of a composition comprising at least one aliphatic-aromatic polyester and at least one additional polymer, in which the additional polymer is selected from the group consisting of polyhydroxy fatty acids, poly(p-dioxanones), polyanhydrides, polyesteramides, polysaccharides and proteins.
2. The composition of spherical microparticles according to embodiment 1, wherein the aliphatic-aromatic polyester is an ester of an aliphatic dihydroxy compound esterified with a composition of aromatic dicarboxylic acid and aliphatic dicarboxylic acid.
3. The composition of spherical microparticles according to embodiment 1 or 2, wherein the aliphatic-aromatic polyester is selected from polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST).
4. The composition of spherical microparticles according to any of embodiments 1 to 3, wherein the composition forming the wall material comprises at least one polymer having a glass transition temperature or a melting point in the range from 45 to 140° C.
5. The composition of spherical microparticles according to any of embodiments 1 to 4, wherein the wall material has a solubility in dichloromethane of at least 50 g/I at 25° C.
6. The composition of spherical microparticles according to any of embodiments 1 to 5,
   wherein the wall material consists of a composition comprising
   30 to 70% by weight of at least one aliphatic-aromatic polyester and also
   30 to 70% by weight of at least one additional polymer selected from the group consisting of polyhydroxy fatty acids, poly(p-dioxanones), polyanhydrides, polyesteramides, polysaccharides and proteins.
7. The composition of spherical microparticles according to any of embodiments 1 to 6, wherein the wall material consists of a composition comprising at least one aliphatic-aromatic polyester and also at least one polyhydroxy fatty acid as additional polymer.
8. The composition of spherical microparticles according to any of embodiments 1 to 7, wherein the at least one polyhydroxy fatty acid is selected from the group consisting of poly(3-hydroxypropionates) (P3HP); poly(2-hydroxybutyrates) (P2HB); copolymers of at least 2 hydroxybutyric acids selected from the group consisting of 2-hydroxybutyric acid, 3-hydroxybutyric acid and 4-hydroxybutyric acid; copolymers of 3-hydroxybutyric acid and 4-hydroxybutyric acid; poly(3-hydroxyvalerates) (P3HV); poly(4-hydroxyvalerates) (P4HV); poly(5-hydroxyvalerates) (P5HV); poly(3-hydroxymethylvalerates) (P3MHV); copolymers of at least 2 hydroxyvaleric acids selected from the group consisting of 3-hydroxyvaleric acid, 4-hydroxyvaleric acid, 5-hydroxyvaleric acid and 3-hydroxymethylvaleric acid; poly(3-hydroxyhexanoates) (P3HHx); poly(4-hydroxyhexanoates) (P4HHx); poly(6-hydroxyhexanoates) (P6HHx); copolymers of at least 2 hydroxyhexanoic acids selected from the group consisting of 3-hydroxyhexanoic acid, 4-hydroxyhexanoic acid and 6-hydroxyhexanoic acid; poly(3-hydroxyoctanoates) (P3HO); poly(4-hydroxyoctanoates) (P4HO); poly(6-hydroxyoctanoates) (P6HO); copolymers of at least 2 hydroxyoctanoic acids selected from the group consisting of 3-hydroxyoctanoic acid, 4-hydroxyoctanoic acid and 6-hydroxyoctanoic acid; poly(3-hydroxyoctanoates) (P3HO); poly(4-hydroxyoctanoates) (P4HO); poly(6-hydroxyoctanoates) (P6HO); copolymers of at least 2 hydroxyoctanoic acids selected from the group consisting of 3-hydroxyoctanoic acid, 4-hydroxyoctanoic acid and 6-hydroxyoctanoic acid; copolyesters of 2-hydroxybutyric acid with at least one monomer selected from the group consisting of 3-hydroxypropionic acid, hydroxyvaleric acids, hydroxyhexanoic acids, hydroxyoctanoic acids and hydroxyoctadecanoic acids; copolyesters of 4-hydroxybutyric acid with 3-hydroxyoctanoic acid [P(4HB-co-3HO)], copolyesters of 3-hydroxybutyric acid with 3-hydroxyoctanoic acid [P(3HB-co-3HO)], copolyesters of 4-hydroxybutyric acid with 3-hydroxyoctadecanoic acid [P(4HB-co-3HOD)], copolyesters of 3-hydroxybutyric acid with 3-hydroxyoctadecanoic acid [P(3HB-co-3HOD)]; copolyesters of hydroxyvaleric acid, especially of 3-hydroxyvaleric acid or 4-hydroxyvaleric acid with at least one monomer selected from the group consisting of 3-hydroxypropionic acid, hydroxyhexanoic acids, hydroxyoctanoic acids and hydroxyoctadecanoic acids; copolyesters of 3-hydroxyhexanoic acid with at least one monomer selected from the group consisting of 3-hydroxypropionic acid, hydroxyoctanoic acid, preferably 3-hydroxyoctanoic acid and hydroxyoctadecanoic acids; and polycaprolactones.
9. The composition of spherical microparticles according to any of embodiments 1 to 8, wherein the polyhydroxy fatty acid is at least one polycaprolactone.
10. The composition of spherical microparticles according to any of embodiments 1 to 9, wherein the wall material consists of a composition comprising at least one further polymer, which is different from the aliphatic-aromatic polyester and from the additional polymer.
11. The composition of spherical microparticles according to embodiment 10, wherein the further polymer is selected from the group consisting of polyacrylate, polyamide, polycarbonate, polystyrene, aliphatic-aliphatic polyester, aromatic-aromatic polyester, polyolefin, polyurea and polyurethane.
12. The composition of spherical microparticles according to embodiment 11, wherein the further polymer is an aliphatic-aliphatic polyester selected from the group consisting of polybutylene succinate adipate, polybutylene succinate, polybutylene sebacate and polybutylene succinate sebacate.
13. The composition of spherical microparticles according to embodiment 10, wherein the further polymer is selected from the group consisting of polyhydroxyacetic acid, PLA copolymers (polylactide and polylactic acid copolymers), PLGA copolymers and polylactic acid.
14. A method for preparing a composition of spherical microparticles according to any of embodiments 1 to 13, wherein
   a) an emulsion is prepared from water or an aqueous solution of a pore former as discontinuous phase and a continuous phase comprising a solution of at least one aliphatic-aromatic polyester and at least one additional polymer selected from the group consisting of polyhydroxy fatty acids, poly(p-dioxanones), polyanhydrides, polyesteramides, polysaccharides and proteins, in a water-immiscible solvent,
   b) the w/o emulsion obtained in a) is emulsified in water in the presence of at least one dispersant to give a w/o/w emulsion having droplets with a mean size of 10-600 μm, and the water-immiscible solvent is removed at a temperature in the range from 20 to 80° C., preferably from 20 to 45° C.,
   c) the spherical microparticles formed in method step b) are separated off and optionally dried.
15. The method for preparing a composition of spherical microparticles according to embodiment 14, wherein the additional polymer is at least one polyhydroxy fatty acid.
16. The method for preparing a composition of spherical microparticles according to either of embodiments 14 to 15, wherein the additional polymer is at least one polyhydroxy fatty acid selected from the group consisting of poly(3-hydroxypropionates) (P3HP); poly(2-hydroxybutyrates) (P2HB); copolymers of at least 2 hydroxybutyric acids selected from the group consisting of 2-hydroxybutyric acid, 3-hydroxybutyric acid and 4-hydroxybutyric acid; copolymers of 3-hydroxybutyric acid and 4-hydroxybutyric acid; poly(3-hydroxyvalerates) (P3HV); poly(4-hydroxyvalerates) (P4HV); poly(5-hydroxyvalerates) (P5HV); poly(3-hydroxymethylvalerates) (P3MHV); copolymers of at least 2 hydroxyvaleric acids selected from the group consisting of 3-hydroxyvaleric acid, 4-hydroxyvaleric acid, 5-hydroxyvaleric acid and 3-hydroxymethylvaleric acid; poly(3-hydroxyhexanoates) (P3HHx); poly(4-hydroxyhexanoates) (P4HHx); poly(6-hydroxyhexanoates) (P6HHx); copolymers of at least 2 hydroxyhexanoic acids selected from the group consisting of 3-hydroxyhexanoic acid, 4-hydroxyhexanoic acid and 6-hydroxyhexanoic acid; poly(3-hydroxyoctanoates) (P3HO); poly(4-hydroxyoctanoates) (P4HO); poly(6-hydroxyoctanoates) (P6HO); copolymers of at least 2 hydroxyoctanoic acids selected from the group consisting of 3-hydroxyoctanoic acid, 4-hydroxyoctanoic acid and 6-hydroxyoctanoic acid; poly(3-hydroxyoctanoates) (P3HO); poly(4-hydroxyoctanoates) (P4HO); poly(6-hydroxyoctanoates) (P6HO); copolymers of at least 2 hydroxyoctanoic acids selected from the group consisting of 3-hydroxyoctanoic acid, 4-hydroxyoctanoic acid and 6-hydroxyoctanoic acid; copolyesters of 2-hydroxybutyric acid with at least one monomer selected from the group consisting of 3-hydroxypropionic acid, hydroxyvaleric acids, hydroxyhexanoic acids, hydroxyoctanoic acids and hydroxyoctadecanoic acids; copolyesters of 4-hydroxybutyric acid with 3-hydroxyoctanoic acid [P(4HB-co-3HO)], copolyesters of 3-hydroxybutyric acid with 3-hydroxyoctanoic acid [P(3HB-co-3HO)], copolyesters of 4-hydroxybutyric acid with 3-hydroxyoctadecanoic acid [P(4HB-co-3HOD)], copolyesters of 3-hydroxybutyric acid with 3-hydroxyoctadecanoic acid [P(3HB-co-3HOD)]; copolyesters of hydroxyvaleric acid, especially of 3-hydroxyvaleric acid or 4-hydroxyvaleric acid with at least one monomer selected from the group consisting of 3-hydroxypropionic acid, hydroxyhexanoic acids, hydroxyoctanoic acids and hydroxyoctadecanoic acids; copolyesters of 3-hydroxyhexanoic acid with at least one monomer selected from the group consisting of 3-hydroxypropionic acid, hydroxyoctanoic acid, preferably 3-hydroxyoctanoic acid and hydroxyoctadecanoic acids; and polycaprolactones.
17. The method for preparing a composition of spherical microparticles according to any of embodiments 14 to 16, wherein the polyhydroxy fatty acid is at least one polycaprolactone.
18. The method for preparing a composition of spherical microparticles according to any of embodiments 14 to 17, wherein the continuous phase prepared in a) comprises a solution of at least one aliphatic-aromatic polyester and at least one additional polymer selected from the group consisting of polyhydroxy fatty acids, poly(p-dioxanones), polyanhydrides, polyesteramides, polysaccharides and proteins and at least one further polymer, in a water-immiscible solvent, wherein the further polymer is different from the aliphatic-aromatic polyester and from the additional polymer.
19. The method for preparing a composition of spherical microparticles according to embodiment 18, wherein the further polymer is selected from the group consisting of polyacrylate, polyamide, polycarbonate, polystyrene, aliphatic-aliphatic polyester, aromatic-aromatic polyester, polyolefin, polyurea and polyurethane.
20. The method for preparing a composition of spherical microparticles according to embodiment 19, wherein the further polymer is an aliphatic-aliphatic polyester selected from the group consisting of polybutylene succinate adipate, polybutylene succinate, polybutylene sebacate and polybutylene succinate sebacate.
21. The method for preparing a composition of spherical microparticles according to embodiment 18, wherein the further polymer is selected from the group consisting of polyhydroxyacetic acid, PLA copolymers (polylactide and polylactic acid copolymers), PLGA copolymers and polylactic acid.
22. The method according to any of embodiments 14 to 21, wherein the water-immiscible solvent is selected from dichloromethane, chloroform, ethyl acetate, n-hexane, cyclohexane, methyl tert-butyl ether, pentane, diisopropyl ether and benzene, or mixtures of these solvents.
23. The method according to any of embodiments 14 to 22, wherein the emulsification to give the w/o/w emulsion in method step b) is effected with a stirrer for a period of 1-30 minutes.
24. The use of the composition of spherical microparticles according to any of embodiments 1 to 13, as carrier substance for filling with at least one aroma chemical.
25. A method for preparing an aroma chemical preparation, wherein the optionally dried composition of spherical microparticles according to any of embodiments 1 to 13 are impregnated with at least one aroma chemical.
26. The method according to embodiment 25, wherein the microparticles are impregnated using a method in which the aroma chemical is present in finely divided form, preferably in the form of droplets.
27. The method according to embodiment 26, wherein the microparticles are sprayed or applied dropwise with an aroma chemical or a solution of at least one aroma chemical.
26. A method according to embodiment 25, wherein the optionally dried composition of spherical microparticles according to any of embodiments 1 to 13 is suspended in a liquid aroma chemical or in a solution of at least one aroma chemical.
28. A method for preparing an aroma chemical preparation, wherein the optionally dried composition of spherical microparticles according to any of embodiments 1 to 13 is suspended in a liquid aroma chemical or in a solution of at least one aroma chemical, and subsequently kept at a temperature in the range from 35 to 200° C., preferably in the range from 40 to 140° C., especially in the range from 45 to 80° C., for a period of 1 minute to 10 hours.
29. An aroma chemical preparation obtainable according to a method according to embodiments 25 to 28.
30. The use of the aroma chemical preparation according to embodiment 29, wherein it is used in an agent selected from perfumes, washing and cleaning agents, cosmetic agents, body care agents, hygiene articles, food, food supplements, scent dispensers or fragrances.
31. An agent comprising a composition of spherical microparticles according to any of embodiments 1 to 13 or an aroma chemical preparation according to embodiment 29, in a proportion by weight of 0.01 to 99.9% by weight, based on the total weight of the composition.
32. The use of the aroma chemical preparation according to embodiment 29 for the controlled release of aroma chemicals.
33. A method for preparing spherical microparticles, wherein
   a) an emulsion is prepared from water or preferably an aqueous solution of a pore former as discontinuous phase and a continuous phase comprising a solution of at least one aliphatic-aromatic polyester and at least one additional polymer selected from the group consisting of polyhydroxy fatty acids, poly(p-dioxanones), polyanhydrides, polyesteramides, polysaccharides and proteins, in a water-immiscible solvent,
   b) the w/o emulsion obtained in a) is emulsified in water in the presence of at least one dispersant to give a w/o/w emulsion having droplets with a mean size of 10-600 μm, and the water-immiscible solvent is removed at a temperature in the range from 20 to 80° C.,
   c) the spherical microparticles formed in method step b) are separated off and optionally dried.
34. The method according to embodiment 33, wherein the aliphatic-aromatic polyester is an ester of an aliphatic dihydroxy compound esterified with a composition of aromatic dicarboxylic acid and aliphatic dicarboxylic acid.
35. The method according to either of embodiments 33 or 34, wherein the aliphatic-aromatic polyester is selected from polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST).
36. The method according to any of embodiments 33 to 35, wherein at least one of the polymers present in the continuous phase of a) has a glass transition temperature or a melting point in the range from 45 to 140° C.
37. The method according to any of embodiments 33 to 36, wherein one of the polymers present in the continuous phase of a) is (partially) crystalline and has a melting point in the range from 45 to 140° C., or is amorphous and has a glass transition temperature in the range from 45 to 140° C.
38. The method according to any of embodiments 33 to 37, wherein the continuous phase prepared in a) comprises at least one polyhydroxy fatty acid as additional polymer.
39. The method according to any of embodiments 33 to 38, wherein the continuous phase prepared in a) comprises, as additional polymer, at least one polyhydroxy fatty acid selected from the group consisting of poly(3-hydroxypropionates) (P3HP); poly(2-hydroxybutyrates) (P2HB); copolymers of at least 2 hydroxybutyric acids selected from the group consisting of 2-hydroxybutyric acid, 3-hydroxybutyric acid and 4-hydroxybutyric acid; copolymers of 3-hydroxybutyric acid and 4-hydroxybutyric acid; poly(3-hydroxyvalerates) (P3HV); poly(4-hydroxyvalerates) (P4HV); poly(5-hydroxyvalerates) (P5HV); poly(3-hydroxymethylvalerates) (P3MHV); copolymers of at least 2 hydroxyvaleric acids selected from the group consisting of 3-hydroxyvaleric acid, 4-hydroxyvaleric acid, 5-hydroxyvaleric acid and 3-hydroxymethylvaleric acid; poly(3-hydroxyhexanoates) (P3HHx); poly(4-hydroxyhexanoates) (P4HHx); poly(6-hydroxyhexanoates) (P6HHx); copolymers of at least 2 hydroxyhexanoic acids selected from the group consisting of 3-hydroxyhexanoic acid, 4-hydroxyhexanoic acid and 6-hydroxyhexanoic acid; poly(3-hydroxyoctanoates) (P3HO); poly(4-hydroxyoctanoates) (P4HO); poly(6-hydroxyoctanoates) (P6HO); copolymers of at least 2 hydroxyoctanoic acids selected from the group consisting of 3-hydroxyoctanoic acid, 4-hydroxyoctanoic acid and 6-hydroxyoctanoic acid; poly(3-hydroxyoctanoates) (P3HO); poly(4-hydroxyoctanoates) (P4HO); poly(6-hydroxyoctanoates) (P6HO); copolymers of at least 2 hydroxyoctanoic acids selected from the group consisting of 3-hydroxyoctanoic acid, 4-hydroxyoctanoic acid and 6-hydroxyoctanoic acid; copolyesters of 2-hydroxybutyric acid with at least one monomer selected from the group consisting of 3-hydroxypropionic acid, hydroxyvaleric acids, hydroxyhexanoic acids, hydroxyoctanoic acids and hydroxyoctadecanoic acids; copolyesters of 4-hydroxybutyric acid with 3-hydroxyoctanoic acid [P(4HB-co-3HO)], copolyesters of 3-hydroxybutyric acid with 3-hydroxyoctanoic acid [P(3HB-co-3HO)], copolyesters of 4-hydroxybutyric acid with 3-hydroxyoctadecanoic acid [P(4HB-co-3HOD)], copolyesters of 3-hydroxybutyric acid with 3-hydroxyoctadecanoic acid [P(3HB-co-3HOD)]; copolyesters of hydroxyvaleric acid, especially of 3-hydroxyvaleric acid or 4-hydroxyvaleric acid with at least one monomer selected from the group consisting of 3-hydroxypropionic acid, hydroxyhexanoic acids, hydroxyoctanoic acids and hydroxyoctadecanoic acids; copolyesters of 3-hydroxyhexanoic acid with at least one monomer selected from the group consisting of 3-hydroxypropionic acid, hydroxyoctanoic acid, preferably 3-hydroxyoctanoic acid and hydroxyoctadecanoic acids; and polycaprolactones.
40. The method according to any of embodiments 33 to 39, wherein the continuous phase prepared in a) comprises at least one polycaprolactone as additional polymer.
41. The method according to any of embodiments 33 to 40, wherein the continuous phase prepared in a) essentially consists of the solution of an aliphatic-aromatic polyester and at least one additional polymer selected from the group consisting of polyhydroxy fatty acids, poly(p-dioxanones), polyanhydrides, polyesteramides, polysaccharides and proteins, in a water-immiscible solvent.
42. The method according to any of embodiments 33 to 41, wherein the ratio of aliphatic-aromatic polyester to additional polymer is 3/7 to 7/3.
43. The method according to any of embodiments 33 to 42, wherein the continuous phase prepared in a) comprises at least one further polymer, which is different from the aliphatic-aromatic polyester and from the additional polymer.
44. The method according to embodiment 43, wherein the further polymer is selected from the group consisting of polyacrylate, polyamide, polycarbonate, polystyrene, aliphatic-aliphatic polyester, aromatic-aromatic polyester, polyolefin, polyurea and polyurethane.
45. The method according to embodiment 44, wherein the further polymer is an aliphatic-aliphatic polyester selected from the group consisting of polybutylene succinate adipate, polybutylene succinate, polybutylene sebacate and polybutylene succinate sebacate.
46. The method according to embodiment 43, wherein the further polymer is selected from the group consisting of polyhydroxyacetic acid, PLA copolymers (polylactide and polylactic acid copolymers), PLGA copolymers and polylactic acid.
47. The method according to any of embodiments 43 to 46, wherein the ratio of aliphatic-aromatic polyester to the sum of additional polymer and further polymer is 3/7 to 7/3.
48. The method according to any of embodiments 33 to 47, wherein the water-immiscible solvent is selected from dichloromethane, chloroform, ethyl acetate, n-hexane, cyclohexane, methyl tert-butyl ether, pentane, diisopropyl ether and benzene, or mixtures of these solvents.
49. The method according to any of embodiments 33 to 48, wherein the emulsification to give the w/o/w emulsion in method step b) is effected with a stirrer for a period of 1-30 minutes.
50. The spherical microparticles obtainable according to a method of embodiments 33 to 29.
51. The use of the spherical microparticles according to any of embodiments 1 to 13 or according to embodiment 50, as carrier substance for filling with at least one aroma chemical.
52. The method according to any of embodiments 33 to 49, wherein subsequently the optionally dried spherical microparticles are impregnated with at least one aroma chemical.
53. The method according to embodiment 52, wherein the microparticles are impregnated using a method in which the aroma chemical is present in finely divided form, preferably in the form of droplets.
54. The method according to embodiment 52 to 53, wherein the microparticles are sprayed or applied dropwise with an aroma chemical or a solution of at least one aroma chemical.
55. The method according to any of embodiments 33 to 52, wherein, subsequently,
   e) the optionally dried spherical microparticles are suspended in a liquid aroma chemical or in a solution of at least one aroma chemical.
56. The method according to embodiment 55, wherein, subsequently,
   f) the microparticles obtained after e) are kept at a temperature in the range from 35 to 200° C., preferably 40 to 140° C., especially in the range from 45 to 80° C., for a period of 1 minute to 10 hours.
57. An aroma chemical preparation obtainable according to any of embodiments 52 to 56.
58. The use of the aroma chemical preparation according to embodiment 57, wherein it is used in an agent selected from perfumes, washing and cleaning agents, cosmetic agents, body care agents, hygiene articles, food, food supplements, scent dispensers or fragrances.
59. An agent comprising a composition of spherical microparticles according to any of embodiments 1 to 13 or an aroma chemical preparation according to embodiment 57, in a proportion by weight of 0.01 to 99.9% by weight, based on the total weight of the composition.
60. The use of the aroma chemical preparation according to embodiment 57 for the controlled release of aroma chemicals.

The term “biodegradable” is understood to mean that the substance in question, the unfilled microparticles here, in the test of OECD Guideline 301B from 1992 (measurement of evolution of CO₂ on composting in a mineral slurry and comparison with the theoretical maximum possible evolution of CO₂) after 28 days and 25° C. undergoes biodegradation of at least 5%, particularly at least 10% and especially at least 20%.

The following related term, spherical microparticles, denotes a spherically formed polymer microparticle (or polymer microsphere). In one embodiment, this may be microcapsules, i.e. particles, in which an outer polymer layer encloses a core that is liquid or gaseous at room temperature.

Fillable spherical microparticles have openings on the surface thereof, such that an exchange of the material inside is possible. In the case of microcapsules, these are holes in the outer polymer layer, often also referred to as microcapsule shell or microcapsule wall. There are however also embodiments with porous spherical microparticles, which have a polymer matrix form. In these cases, this is a connected porous network that has openings at the surface of the microparticle.

Furthermore, there are embodiments of microparticles, the morphology of which has both.

The microparticles are formed by removal of the solvent in a w/o/w emulsion. In the first step, an emulsion of water droplets or droplets of the aqueous pore former solution is formed in the polyester solution. This w/o emulsion is in turn emulsified in water and the water-immiscible solvent is removed. By removing the solvent of the polyester, the latter becomes insoluble and becomes deposited at the surface of the water droplets or the aqueous pore former droplets. During this wall forming process, the pores are simultaneously formed, advantageously brought about by the pore former.

Pore formers are, for example, compounds that release gas under the process conditions of step b).

Pore formers are for example gas-releasing agents preferably selected from ammonium carbonate, sodium carbonate, ammonium hydrogencarbonate, ammonium sulfate, ammonium oxalate, sodium hydrogencarbonate, ammonium carbamate and sodium carbamate.

Further suitable pore formers are water-soluble low molecular weight compounds that create an osmotic pressure. Removal of the water-insoluble solvent, on account of the concentration gradient that exists between the inner aqueous droplets with pore former and the outer aqueous disperse phase, builds up a concentration gradient which leads to migration of the water in the direction of the inner droplets and hence to the formation of pores. Such pore formers are preferably selected from sugars such as monosaccharides, disaccharides, oligosaccharides and polysaccharides, urea, inorganic alkali metal salts such as sodium chloride and inorganic alkaline earth metal salts such as magnesium sulfate and calcium chloride. Particular preference is given to glucose and sucrose and urea.

Furthermore, polymers that are soluble in both phases, such as polyethylene glycol (PEG) and polyvinylpyrrolidone (PVP) are suitable as pore formers. Since these polymers are soluble in both phases, they migrate from the aqueous phase into the oil phase owing to diffusion.

The processes for producing the spherical microparticles always lead to a population of microparticles, and therefore the term “composition of spherical microparticles” is also used.

The inventive microparticles have a mean particle diameter of D[4,3] from 10 to 600 μm (volume-weighted average, determined by means of light scattering). According to a preferred embodiment, the mean particle diameter D[4,3] is 10 to <100, preferably to 30 μm. According to a likewise preferred embodiment, the mean particle diameter D[4,3] is 100-500 am.

The inventive microparticles have at least 10 pores at their surface, preferably at least 20 pores, the diameter of which is in the range from 1/5000 to 1/5 of the mean particle diameter D[4,3], and furthermore the diameter of each of these pores is at least 20 nm. The microparticles preferably have on average at least 10 pores, preferably at least 20 pores, the diameter of which is in the range from 1/500 to 1/5 of the mean particle diameter D[4,3], and furthermore the diameter of each of these pores is at least 20 nm. The microparticles preferred according to one embodiment, of mean particle diameter 100-500 μm, preferably have pores having a mean diameter D[4,3] in the range from 1/500 to 1/100 of the mean particle diameter. In each case, those microparticles of the composition of spherical microparticles whose particle diameter does not deviate from the mean particle diameter D[4,3] by more than 20% are taken into consideration. Of these, at least 80% meet the required number of pores at the particle surface.

According to the invention, an aliphatic-aromatic polyester is used. This term is understood to mean the esters based on aromatic dicarboxylic acids and aliphatic dihydroxy compounds. The aromatic dicarboxylic acids may also be used in a mixture with aliphatic dicarboxylic acids here. Aliphatic-aromatic polyesters are preferably polyesters based on aliphatic and aromatic dicarboxylic acids with aliphatic dihydroxy compound, what are referred to as semiaromatic polyesters. These polymers may be present individually or in the mixtures thereof.

The aliphatic-aromatic polyesters used according to the invention preferably have a glass transition temperature (determined using differential scanning calorimetry (DSC), DIN EN ISO 11357) or a melting point in the range from 45 to 140° C.

According to the invention, aliphatic-aromatic polyester is also understood to mean polyester derivatives of these aliphatic-aromatic polyesters, such as polyether esters, polyester amides or polyether ester amides and polyester urethanes (see EP application no. 10171237.0). The suitable aliphatic-aromatic polyesters include linear, non-chain-extended polyesters (WO 92/09654). Preference is given to chain-extended and/or branched aliphatic-aromatic polyesters. The latter are known from WO 96/15173 to 15176, 21689 to 21692, 25446, 25448 or WO 98/12242, which are hereby explicitly incorporated by reference. Likewise considered are mixtures of different aliphatic-aromatic polyesters. Interesting recent developments are based on renewable raw materials (see WO-A 2006/097353, WO-A 2006/097354 and also WO 2010/034710).

Particularly preferred aliphatic-aromatic polyesters include polyesters comprising as essential components:

A) an acid component formed of
a1) 30 to 99 mol % of at least one aliphatic dicarboxylic acid or the ester-forming derivatives thereof or mixtures thereof
a2) 1 to 70 mol % of at least one aromatic dicarboxylic acid or the ester-forming derivative thereof or mixtures thereof, and
B) at least one diol component selected from C to C₁₂ alkanediols
   and
C) optionally a component selected from
   c1) a compound having at least three groups capable of ester formation,
   c2) a diisocyanate or polyisocyanate,
   c3) a diepoxide or polyepoxide,

Aliphatic dicarboxylic acids and the ester-forming derivatives thereof (a1) that are generally considered are those having 2 to 18 carbon atoms, preferably 4 to 10 carbon atoms. They may be either linear or branched. However, it is also possible in principle to employ dicarboxylic acids having a greater number of carbon atoms, for example having up to 30 carbon atoms.

Examples include: oxalic acid, malonic acid, succinic acid, 2-methylsuccinic acid, glutaric acid, 2-methylglutaric acid, 3-methylglutaric acid, α-ketoglutaric acid, adipic acid, pimelic acid, azelaic acid, sebacic acid, brassylic acid, fumaric acid, 2,2-dimethylglutaric acid, suberic acid, diglycolic acid, oxaloacetic acid, glutamic acid, aspartic acid, itaconic acid and maleic acid. These dicarboxylic acids or the ester-forming derivatives thereof may be used individually or as a mixture of two or more thereof.

It is preferable to employ succinic acid, adipic acid, azelaic acid, sebacic acid, brassylic acid or their respective ester-forming derivatives or mixtures thereof. It is particularly preferable to employ succinic acid, adipic acid or sebacic acid or their respective ester-forming derivatives or mixtures thereof. Succinic acid, azelaic acid, sebacic acid and brassylic acid additionally have the advantage that they are obtainable from renewable raw materials.

Preference is given to the following aliphatic-aromatic polyesters: polybutylene azelate-co-butylene terephthalate (PBAzeT), polybutylene brassylate-co-butylene terephthalate (PBBrasT), and especially preferably: polybutylene adipate terephthalate (PBAT), polybutylene sebacate terephthalate (PBSeT) or polybutylene succinate terephthalate (PBST).

The aromatic dicarboxylic acids or the ester-forming derivatives thereof (a2) may be used individually or as a mixture of two or more thereof. Particular preference is given to using terephthalic acid or the ester-forming derivatives thereof such as dimethyl terephthalate.

Generally, the diols (B) are selected from branched or linear alkanediols having 2 to 12 carbon atoms, preferably 4 to 6 carbon atoms, or cycloalkanediols having 5 to 10 carbon atoms.

Examples of suitable alkanediols are ethylene glycol, 1,2-propanediol, 1,3-propanediol, 1,2-butanediol, 1,4-butanediol, 1,5-pentanediol, 2,4-dimethyl-2-ethylhexane-1,3-diol, 2,2-dimethyl-1,3-propanediol, 2-ethyl-2-butyl-1,3-propanediol, 2-ethyl-2-isobutyl-1,3-propanediol, 2,2,4-trimethyl-1,6-hexanediol, especially ethylene glycol, 1,3-propanediol, 1,4-butanediol and 2,2-dimethyl-1,3-propanediol (neopentyl glycol); examples of cycloalkanediols are cyclopentanediol, 1,4-cyclohexanediol, 1,2-cyclohexanedimethanol, 1,3-cyclohexanedimethanol, 1,4-cyclohexanedimethanol and 2,2,4,4-tetramethyl-1,3-cyclobutanediol. The aliphatic-aromatic polyesters may also comprise mixtures of different alkanediols condensed in. Particular preference is given to butane-1,4-diol, especially in combination with adipic acid or sebacic acid as component a1), and propane-1,3-diol, especially in combination with sebacic acid as component a1). 1,3-Propanediol also has the advantage that it is obtainable as a renewable raw material.

The preferred aliphatic-aromatic polyesters are characterized by a molecular weight (Mn) in the range from 1000 to 100 000, especially in the range from 9000 to 75 000 g/mol, preferably in the range from 10 000 to 50 000 g/mol.

In accordance with the invention, the composition of the wall material comprises at least one aliphatic-aromatic polyester and at least one additional polymer, in which the additional polymer is selected from the group consisting of polyhydroxy fatty acids, poly(p-dioxanones), polyanhydrides, polyesteramides, polysaccharides and proteins.

In a preferred embodiment, the additional polymer is at least one polyhydroxy fatty acid, preferably at least one polycaprolactone.

By definition, the additional polymer is a polymer different from the aliphatic-aromatic polyester.

### Polyhydroxy Fatty Acids,

Polyhydroxy fatty acids to be used in accordance with the invention are those which comprise monomers having a chain length in the polymer backbone of at least 3 carbon atoms. Polylactic acid and polyhydroxyacetic acid are therefore not polyhydroxy fatty acids in the context of the invention.

In accordance with the invention, preference is given to using at least one polyhydroxy fatty acid comprising repeating monomer units of the formula (1)

[—O—CHR—(CH₂)ₘ—CO—] (1)

where R is hydrogen or a linear or branched alkyl group having 1 to 20, preferably 1 to 16 carbon atoms, preferably 1 to 6 carbon atoms and m=numbers from 1 to 18, preferably 1, 2, 3, 4, 5 and 6; and/or homopolymers of 2-hydroxybutyric acid.

In accordance with the invention, preference is given to using at least one polyhydroxy fatty acid comprising repeating monomer units of the formula (1)

[—O—CHR—(CH₂)ₘ—CO—] (1)

where R is hydrogen or a linear or branched alkyl group having 1 to 20, preferably 1 to 16 carbon atoms, preferably 1 to 6 carbon atoms and m=numbers from 1 to 18, preferably 1, 2, 3, 4, 5 and 6; and/or homopolymers of 2-hydroxybutyric acid, excluding poly(4-hydroxybutyrates) and poly(3-hydroxybutyrates), furthermore copolyesters of the aforementioned hydroxybutyrates with 3-hydroxyvalerates (P(3HB)-co-P(3HV)) or 3-hydroxyhexanoate.

The polyhydroxy fatty acids comprise homopolymers (synonym homopolyesters), i.e. polyhydroxy fatty acids consisting of identical hydroxy fatty acid monomers and also copolymers (synonym copolyesters), i.e. polyhydroxy fatty acids consisting of different hydroxy fatty acid monomers.

The polyhydroxy fatty acids may be used individually or in the form of any mixtures.

Polyhydroxy fatty acids in the context of this invention especially have molecular weights M_{w} of 5000 to 1 000 000, in particular 30 000 to 1 000 000, particularly 70 000 to 1 000 000, preferably 100 000 to 1000 000 or 300 000 to 600 000 and/or melting points in the range of 100 to 190° C.

In one embodiment of the invention, the at least one polyhydroxy fatty acid is selected from the group consisting of
poly(3-hydroxypropionates) (P3HP);
polyhydroxybutyrates (PHB);
polyhydroxyvalerates (PHV);
polyhydroxyhexanoates (PHHx);
polyhydroxyoctanoates (PHO);
polyhydroxyoctadecanoates (PHOD);
copolyesters of hydroxybutyric acid with at least one monomer selected from the group consisting of 3-hydroxypropionic acid, hydroxyvaleric acids, hydroxyhexanoic acids, hydroxyoctanoic acids and hydroxyoctadecanoic acids;
copolyesters of hydroxyvaleric acid with at least one monomer selected from the group consisting of 3-hydroxypropionic acid, hydroxyhexanoic acids, hydroxyoctanoic acids and hydroxyoctadecanoic acids;
copolyesters of hydroxyhexanoic acid with at least one monomer selected from the group consisting of 3-hydroxypropionic acid, hydroxyoctanoic acid and hydroxyoctadecanoic acid;
polycaprolactones.

Suitable polyhydroxybutyrates (PHB) may be selected from the group consisting of poly(2-hydroxybutyrates) (P2HB), poly(3-hydroxybutyrates) (P3HB), poly(4-hydroxybutyrates) (P4HB) and copolymers of at least 2 hydroxybutyric acids selected from the group consisting of 2-hydroxybutyric acid, 3-hydroxybutyric acid and 4-hydroxybutyric acid. Further suitable are copolymers of 3-hydroxybutyric acid and 4-hydroxybutyric acid. These copolymers are characterized by the following abbreviations: [P(3HB-co-4HB)], where 3HB is 3-hydroxybutyrate and 4HB is 4-hydroxybutyrates.

Poly(3-hydroxybutyrates) are marketed for example by PHB Industrial under the brand Biocycle® and by Tianan under the name Enmat. Poly-3-hydroxybutyrate-co-4-hydroxybutyrates are known from Metabolix in particular. They are sold under the trade name Mirel®.

Suitable polyhydroxyvalerates (PHV) may be selected from the group consisting of homopolymers of 3-hydroxyvaleric acid [=poly(3-hydroxyvalerates) (P3HV)], homopolymers of 4-hydroxyvaleric acid [=poly(4-hydroxyvalerates) (P4HV)]; homopolymers of 5-hydroxyvaleric acid [=poly(5-hydroxyvalerates) (P5HV)]; homopolymers of 3-hydroxymethylvaleric acid [=poly(3-hydroxymethylvalerates) (P3MHV)]; copolymers of at least 2 hydroxyvaleric acids selected from the group consisting of 3-hydroxyvaleric acid, 4-hydroxyvaleric acid, 5-hydroxyvaleric acid and 3-hydroxymethylvaleric acid.

Suitable polyhydroxyhexanoates (PHHx) may be selected from the group consisting of poly(3-hydroxyhexanoates) (P3HHx), poly(4-hydroxyhexanoates) (P4HHx), poly(6-hydroxyhexanoates) (P6HHx) and copolymers of at least 2 hydroxyhexanoic acids selected from the group consisting of 3-hydroxyhexanoic acid, 4-hydroxyhexanoic acid and 6-hydroxyhexanoic acid.

Suitable polyhydroxyoctanoates (PHO) may be selected from the group consisting of poly(3-hydroxyoctanoates) (P3HO), poly(4-hydroxyoctanoates) (P4HO), poly(6-hydroxyoctanoates) (P6HO) and copolymers of at least 2 hydroxyoctanoic acids selected from the group consisting of 3-hydroxyoctanoic acid, 4-hydroxyoctanoic acid and 6-hydroxyoctanoic acid.

Suitable copolyesters of hydroxybutyric acid with at least one monomer selected from the group consisting of 3-hydroxypropionic acid, hydroxyvaleric acids, hydroxyhexanoic acids, hydroxyoctanoic acids and hydroxyoctadecanoic acids may be selected from the group consisting of
copolyesters of 4-hydroxybutyric acid with 3-hydroxyvaleric acid [P(4HB-co-3HV)]
copolyesters of 3-hydroxybutyric acid with 3-hydroxyvaleric acid [P(3HB-co-3HV)]
copolyesters of 4-hydroxybutyric acid with 3-hydroxyhexanoic acid [P(4HB-co-3HHx)]
copolyesters of 3-hydroxybutyric acid with 3-hydroxyhexanoic acid [P(3HB-co-3HHx)]
copolyesters of 4-hydroxybutyric acid with 3-hydroxyoctanoic acid [P(4HB-co-3HO)] and
copolyesters of 3-hydroxybutyric acid with 3-hydroxyoctanoic acid [P(3HB-co-3HO)]
copolyesters of 4-hydroxybutyric acid with 3-hydroxyoctadecanoic acid [P(4HB-co-3HOD)] and
copolyesters of 3-hydroxybutyric acid with 3-hydroxyoctadecanoic acid [P(3HB-co-3HOD)]

Preference is given to using poly-3-hydroxybutyrate-co-3-hydroxyhexanoate having a 3-hydroxyhexanoate proportion of 1 to 20 and preferably of 3 to 15 mol % based on the total amount of polyhydroxy fatty acid. Such poly-3-hydroxybutyrate-co-3-hydroxyhexanoates [P(3HB-co-3HHx] are known from Kaneka and are commercially available under the trade names Aonilex™ X131A and Aonilex™ X151A.

Suitable copolyesters of hydroxyvaleric acid are preferably copolyesters of 4-hydroxyvaleric acid and/or 3-hydroxyvaleric acid with at least one monomer selected from the group consisting of 3-hydroxypropionic acid, hydroxyhexanoic acids, hydroxyoctanoic acids, especially 3-hydroxyoctanoic acid and hydroxyoctadecanoic acids.

Suitable copolyesters of hydroxyhexanoic acid are preferably copolyesters of 3-hydroxyhexanoic acid with at least one monomer selected from the group consisting of 3-hydroxypropionic acid and hydroxyoctanoic acid, preferably 3-hydroxyoctanoic acid and hydroxyoctadecanoic acids.

Polycaprolactones (PCL) refer to polyesters obtainable by ring-opening polymerization of epsilon-caprolactone (ε-caprolactone). Polycaprolactones are therefore polyhydroxy fatty acids with repeating monomer units of the general formula (1) [—O—CHR—(CH₂)ₘ—CO—], in which m=4 and R=hydrogen. In the context of the invention, the term polycaprolactone is understood to mean both homopolymers of epsilon-caprolactone and copolymers of epsilon-caprolactone. Suitable copolymers are, for example, copolymers of epsilon-caprolactone with monomers selected from the group consisting of lactic acid, lactide, hydroxyacetic acid and glycolide.

Polycaprolactones are marketed, for example, by Perstorp under the brand name Capa™ or by Daicel under the brand name Celgreen™.

In a preferred embodiment, the at least one polyhydroxy fatty acid, is a polycaprolactone.

In one embodiment of the invention, the at least one polyhydroxy fatty acid is selected from the group consisting of
poly(3-hydroxypropionates) (P3HP);
polyhydroxybutyrates (PHB);
polyhydroxyvalerates (PHV);
polyhydroxyhexanoates (PHHx);
polyhydroxyoctanoates (PHO);
polyhydroxyoctadecanoates (PHOD);
copolyesters of hydroxybutyric acid with at least one monomer selected from the group consisting of 3-hydroxypropionic acid, hydroxyvaleric acids, hydroxyhexanoic acids, hydroxyoctanoic acids and hydroxyoctadecanoic acids;
copolyesters of hydroxyvaleric acid with at least one monomer selected from the group consisting of 3-hydroxypropionic acid, hydroxyhexanoic acids, hydroxyoctanoic acids and hydroxyoctadecanoic acids;
copolyesters of hydroxyhexanoic acid with at least one monomer selected from the group consisting of 3-hydroxypropionic acid, hydroxyoctanoic acid and hydroxyoctadecanoic acid;
polycaprolactones
   excluding poly(4-hydroxybutyrates) and poly(3-hydroxybutyrates), furthermore copolyesters of the aforementioned hydroxybutyrates with 3-hydroxyvalerates (P(3HB)-co-P(3HV)) or 3-hydroxyhexanoate.

In one embodiment of the invention, the at least one polyhydroxy fatty acid is selected from the group consisting of poly(3-hydroxypropionates) (P3HP); poly(2-hydroxybutyrates) (P2HB); copolymers of at least 2 hydroxybutyric acids selected from the group consisting of 2-hydroxybutyric acid, 3-hydroxybutyric acid and 4-hydroxybutyric acid; copolymers of 3-hydroxybutyric acid and 4-hydroxybutyric acid; poly(3-hydroxyvalerates) (P3HV); poly(4-hydroxyvalerates) (P4HV); poly(5-hydroxyvalerates) (P5HV); poly(3-hydroxymethylvalerates) (P3MHV); copolymers of at least 2 hydroxyvaleric acids selected from the group consisting of 3-hydroxyvaleric acid, 4-hydroxyvaleric acid, 5-hydroxyvaleric acid and 3-hydroxymethylvaleric acid; poly(3-hydroxyhexanoates) (P3HHx); poly(4-hydroxyhexanoates) (P4HHx); poly(6-hydroxyhexanoates) (P6HHx); copolymers of at least 2 hydroxyhexanoic acids selected from the group consisting of 3-hydroxyhexanoic acid, 4-hydroxyhexanoic acid and 6-hydroxyhexanoic acid; poly(3-hydroxyoctanoates) (P3HO); poly(4-hydroxyoctanoates) (P4HO); poly(6-hydroxyoctanoates) (P6HO); copolymers of at least 2 hydroxyoctanoic acids selected from the group consisting of 3-hydroxyoctanoic acid, 4-hydroxyoctanoic acid and 6-hydroxyoctanoic acid; poly(3-hydroxyoctanoates) (P3HO); poly(4-hydroxyoctanoates) (P4HO); poly(6-hydroxyoctanoates) (P6HO); copolymers of at least 2 hydroxyoctanoic acids selected from the group consisting of 3-hydroxyoctanoic acid, 4-hydroxyoctanoic acid and 6-hydroxyoctanoic acid; copolyesters of 2-hydroxybutyric acid with at least one monomer selected from the group consisting of 3-hydroxypropionic acid, hydroxyvaleric acids, hydroxyhexanoic acids, hydroxyoctanoic acids and hydroxyoctadecanoic acids; copolyesters of 4-hydroxybutyric acid with 3-hydroxyoctanoic acid [P(4HB-co-3HO)], copolyesters of 3-hydroxybutyric acid with 3-hydroxyoctanoic acid [P(3HB-co-3HO)], copolyesters of 4-hydroxybutyric acid with 3-hydroxyoctadecanoic acid [P(4HB-co-3HOD)], copolyesters of 3-hydroxybutyric acid with 3-hydroxyoctadecanoic acid [P(3HB-co-3HOD)]; copolyesters of hydroxyvaleric acid, especially of 3-hydroxyvaleric acid or 4-hydroxyvaleric acid with at least one monomer selected from the group consisting of 3-hydroxypropionic acid, hydroxyhexanoic acids, hydroxyoctanoic acids and hydroxyoctadecanoic acids; copolyesters of 3-hydroxyhexanoic acid with at least one monomer selected from the group consisting of 3-hydroxypropionic acid, hydroxyoctanoic acid, preferably 3-hydroxyoctanoic acid and hydroxyoctadecanoic acids; and polycaprolactones.

### Poly(p-Dioxanones) (PPDO)

Poly-p-dioxanones (poly-1,4-dioxan-2-one) refer to poly(ether-esters) obtainable by ring-opening polymerization of 1,4-dioxan-2-one.

In the context of the present invention, the term poly(p-dioxanones) are understood to mean homopolymers of 1,4-dioxan-2-one, which have the general structural unit [—O—CH₂—CH₂—O—CH₂—CO-]n. In the context of the present invention, the term poly(p-dioxanones) is also understood to mean copolymers of 1,4-dioxan-2-one with lactone monomers. Particularly suitable are copolymers of 1,4-dioxan-2-one with at least one further monomer selected from the group consisting of glycolide, lactide and epsilon-caprolactone.

### Polyanhydrides

Polyanhydrides refer to polymers having the general structural unit

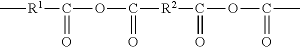

as characteristic base units of the main chain. R¹ and R² can be the same or different aliphatic or aromatic radicals.

Suitable polyanhydrides are described in Kumar et al, Adv. Drug Delivery Reviews 54 (2002), pp. 889-910. Particularly suitable are the polyanhydrides described in Kumar et al. Adv. Drug Delivery Reviews 54 (2002), on p. 897, which is fully incorporated here by way of reference. In one embodiment of the invention, the polyanhydride is selected from the group of aliphatic polyanhydrides, especially from the group consisting of polysebacic acid and polyadipic acid.

### Polyesteramides

Polyesteramides are copolymers of polyamides and polyesters and thus polymers bearing both amide and ester functions. Suitable polyesteramides are particularly polyesteramides obtained by condensation of ε-caprolactam, adipic acid and 1,4-butanediol and polyesteramides obtained by condensation of adipic acid, 1,4-butanediol, diethylene glycol and hexamethylene diamines. Polyesteramides are marketed, for example, under the trade name BAK™ from Bayer, such as BAK™1095 or BAK™ 2195 for example.

### Polysaccharides

Polysaccharides are macromolecules in which a relatively large number of sugar residues are glycosidically linked to one another. Suitable polysaccharides in accordance with the invention are polysaccharides having a solubility in dichloromethane at 25° C. of at least 50 g/1.

In the context of the invention, polysaccharides also include derivatives thereof if they have a solubility in dichloromethane at 25° C. of at least 50 g/1.

Suitable polysaccharides in accordance with the invention are preferably selected from the group consisting of modified starches such as, in particular, starch ethers and esters, cellulose derivatives such as, in particular, cellulose esters and cellulose ethers, chitin derivatives, chitosan derivatives.

Cellulose derivatives generally refer to celluloses chemically modified by polymer-analogous reactions. They comprise both products in which exclusively the hydroxyl hydrogen atoms of the glucose units of the cellulose have been substituted by organic or inorganic groups and those in which formally an exchange of the entire hydroxyl group has been effected (e.g. desoxycelluloses). Also products which are obtained from intramolecular elimination of water (anhydrocelluloses), oxidation reactions (aldehyde-, keto- and carboxycelluloses) or cleavage of the C₂,C₃-carbon bond of the glucose units (dialdehyde- and dicarboxycelluloses) are counted as cellulose derivatives. Finally, cellulose derivatives are also accessible by reactions such as crosslinking or graft copolymerization reactions. Since for all these reactions to some extent a multiplicity of reagents can be used and, in addition, the degree of substitution and polymerization of the cellulose derivatives obtained can be varied, an extensive palette of soluble and insoluble cellulose derivatives having markedly differing properties is known.

Suitable cellulose ethers are, for example, methyl cellulose, ethyl cellulose, hydroxyethyl cellulose, hydroxypropyl cellulose and hydroxypropylmethyl cellulose.

Suitable cellulose ethers are methylhydroxy-(C₁-C₄)-alkylcelluloses. Methyl hydroxy(C₁-C₄)alkyl celluloses are understood to mean methyl hydroxy(C₁-C₄)alkyl celluloses of a wide variety of degrees of methylation and also degrees of alkoxylation.

The preferred methyl hydroxy(C₁-C₄)alkyl celluloses have an average degree of substitution DS of 1.1 to 2.5 and a molar degree of substitution MS of 0.03 to 0.9.

Suitable methyl hydroxy(C₁-C₄)alkyl celluloses are for example methyl hydroxyethyl cellulose or methyl hydroxypropyl cellulose.

Suitable cellulose esters are, for example, the esters of cellulose with C₂-C₄ monocarboxylic acids such as cellulose acetate (commerically available from Eastmann CA-398-3), cellulose butyrate, cellulose acetobutyrate, cellulose propionate and cellulose acetopropionate. Cellulose esters are obtainable in a wide variety of degrees of polymerization and substitution.

### Proteins

Proteins to be used in accordance with the invention comprise polypeptides (acid amide-like condensation products of amino acids linked by peptide bonds) and derivatives thereof having a solubility in dichloromethane at 25° C. of at least 50 g/1. They polypeptides may be of natural or synthetic origin.

Preferably, at least one of the polymers contained in the continuous phase of a) has a glass transition temperature or a melting point in the range from 45 to 140° C. If the polymer has a melting point, i.e. is (partially) crystalline, it preferably has a melting point in the range from 45 to 140° C. If the polymer is amorphous, it preferably has a glass transition temperature in the range from 45 to 140° C.

In a preferred embodiment, the continuous phase prepared in a) consists essentially of the solution of an aliphatic-aromatic polyester and the at least one additional polymer in a water-immiscible solvent. The continuous phase more preferably consists to an extent of at least 95% by weight, especially to an extent of at least 99% by weight, based on the continuous phase, of the solution of an aliphatic-aromatic polyester and the at least one additional polymer in a water-immiscible solvent.

In a likewise preferred embodiment of the method, the continuous phase prepared in a) comprises the aliphatic-aromatic polyester and the at least one additional polymer in a ratio from 3/7 to 7/3.

In a further embodiment of the method, the continuous phase prepared in a) comprises at least one further dissolved polymer.

By definition, the further polymer is a polymer different from the aliphatic-aromatic polyester and different from the additional polymer.

In this embodiment, the continuous phase prepared in a) thus comprises at least one aliphatic-aromatic polyester, at least one additional polymer selected from the group consisting of polyhydroxy fatty acids, poly(p-dioxanones), polyanhydrides, polyesteramides, polysaccharides and proteins and also at least one further polymer.

Further polymers that are not aliphatic-aromatic polyesters or the additional polymers that may for example be mentioned are polyacrylate, polyamide, polycarbonate, polystyrene, aliphatic-aliphatic polyesters, aromatic-aromatic polyesters, polyolefin, polyurea and polyurethane.

In one embodiment of the invention, the further polymer used is at least one polymer selected from the group consisting of polyacrylate, polyamide, polycarbonate, polystyrene, aliphatic-aliphatic polyester, aromatic-aromatic polyester, polyolefin, polyurea and polyurethane.

Suitable polyurethanes are particularly those of which the diol component consists of polyhydroxy fatty acids, PLA or aliphatic-aromatic polyesters.

Aliphatic-aliphatic polyesters are understood to mean polyesters based on aliphatic dicarboxylic acids and aliphatic dihydroxyl compounds, and polyesters based on mixtures of aliphatic dicarboxylic acids with aliphatic dicarboxylic acids and aliphatic dihydroxyl compounds.

Examples of aliphatic carboxylic acids which are suitable for the preparation of aliphatic-aliphatic polyesters are the aliphatic dicarboxylic acids mentioned under (a1), especially those having 2 to 18 carbon atoms, preferably 4 to 10 carbon atoms. Preference is given to aliphatic-aliphatic polyesters in which the aliphatic dicarboxylic acid is selected from succinic acid, adipic acid, azelaic acid, sebacic acid, brassylic acid and mixtures thereof. Particular preference is given to succinic acid, adipic acid and sebacic acid and mixtures thereof. To prepare the aliphatic-aliphatic polyesters, instead of the dicarboxylic acids, their respective ester-forming derivatives or mixtures thereof with the dicarboxylic acids may also be used.

Examples of aliphatic diols which are suitable for the preparation of the aliphatic-aliphatic polyesters are the diols mentioned as component (B), for example branched or linear alkanediols having 2 to 12 carbon atoms, preferably 4 to 6 carbon atoms, or cycloalkanediols having 5 to 10 carbon atoms. Examples of suitable alkanediols are ethylene glycol, 1,2-propanediol, 1,3-propanediol, 1,2-butanediol, 1,4-butanediol, 1,5-pentanediol, 2,4-dimethyl-2-ethylhexane-1,3-diol, 2,2-dimethyl-1,3-propanediol, 2-ethyl-2-butyl-1,3-propanediol, 2-ethyl-2-isobutyl-1,3-propanediol, 2,2,4-trimethyl-1,6-hexanediol, especially ethylene glycol, 1,3-propanediol, 1,4-butanediol and 2,2-dimethyl-1,3-propanediol (neopentyl glycol). Examples of cycloalkanediols are cyclopentanediol, 1,4-cyclohexanediol, 1,2-cyclohexanedimethanol, 1,3-cyclohexanedimethanol, 1,4-cyclohexanedimethanol and 2,2,4,4-tetramethyl-1,3-cyclobutanediol. The aliphatic-aliphatic polyesters may also comprise mixtures of different alkanediols condensed in. Particular preference is given to 1,4-butanediol, especially in combination with one or two aliphatic dicarboxylic acids selected from succinic acid, adipic acid and sebacic acid, as component a1).

Examples of particularly preferred aliphatic-aliphatic polyesters are polybutylene succinate adipate, polybutylene succinate, polybutylene sebacate, polybutylene succinate sebacate.

The preferred aliphatic-aliphatic polyesters often have a molecular weight Mn in the range from 1000 to 100 000 g/mol, particularly in the range from 2000 to 75 000 g/mol, especially in the range from 5000 to 50 000 g/mol.

In one embodiment of the invention, the further polymer used is a polymer selected from the group consisting of polyhydroxyacetic acid, PLA copolymers (polylactide and polylactic acid copolymers), PLGA copolymers and polylactic acid. Preferred PLGA copolymers are polylactide copolymers.

Polylactic acid having a molecular weight of 30 000 to 120 000 Dalton and a glass transition temperature (T_{g}) in the range from 50 to 65° C. is particularly suitable. Most particular preference is given to using amorphous polylactic acid, the D-lactic acid proportion of which is greater than 9%.

In accordance with the invention, preference is given to mixtures of an aliphatic-aromatic polyester with the additional polymer, having a proportion by weight of the aromatic-aliphatic polyester of 20 to 99% by weight (based on the total weight of aliphatic-aromatic polyester and additional polymer). Preferably, the proportion of the aliphatic-aromatic polyester is 25 to 80% by weight, preferably 30 to 70% by weight of the total weight.

If the mixture, in addition to the aliphatic-aromatic polyester and the additional polymer, comprises a further polymer, preference is given to mixtures having a proportion by weight of aromatic-aliphatic polyester of 20 to 99% by weight, preferably 25 to 80% by weight, preferably 30 to 70% by weight (based on the total weight of the aliphatic-aromatic polyester, additional polymer and further polymer).

Preference is given to mixtures of an aliphatic-aromatic polyester with an additional polymer, in which the melting point of the aliphatic-aromatic polyester is at least 10 K, preferably at least 20 K, above the melting point of the additional polymer, or the glass transition temperature of the aliphatic-aromatic polyester is at least 10 K, preferably at least 20 K, above the glass transition temperature of the additional polymer. If the additional polymer is an amorphous compound, then the melting point of the aliphatic-aromatic polyester is at least 10 K, preferably at least 20 K, above the glass transition temperature of the additional polymer.

The composition of microparticles is prepared according to the double emulsion method.

### Method Step a)

Here, the aliphatic-aromatic polyester and the additional polymer and also optionally the further polymer are dissolved in a water-immiscible solvent.

Water-immiscible means that the solvent has a solubility in water, at a temperature of 20° C. and a pressure of 1 bar, of ≤90 g/l. Furthermore, the water-immiscible solvent preferably has a boiling point of at least 30° C.

According to the general knowledge of those skilled in the art, solvents are chemically inert to the substances to be dissolved therein; that is to say, they merely serve for dilution or dissolution. Free radically-polymerizable monomers are not solvents in the context of the invention.

Preference is given to aprotic non-polar and aprotic polar solvents or solvent mixtures, which have a water solubility of <90 g/I (at 20° C.). Preferred solvents are for example dichloromethane, chloroform, ethyl acetate, n-hexane, cyclohexane, methyl-tert-butyl ether, pentane, diisopropyl ether and benzene, or mixtures of two or more of these solvents with one another. Dichloromethane is particularly preferred.

Furthermore, solvent mixtures which form an azeotrope, the boiling point of which is in the range from 20 to 80° C., are suitable. One example is the azeotrope of hexane and methyl ethyl ketone (MEK) in the weight ratio of 72:28.

In general, the polyester, the additional polymer and optionally the further polymer is used as a 1% to 50% by weight solution in the water-immiscible solvent. Preferably, the polymer solution thus prepared is a 2% to 30% by weight, especially 5% to 20% by weight, solution in the water-immiscible solvent.

In accordance with the invention, an emulsion formed of a solution of at least one aliphatic-aromatic polyester and the at least one additional polymer is selected. Preference is given to selecting an emulsion formed of a solution of at least one aliphatic-aromatic polyester and the at least one additional polymer and the at least one further polymer. The solution used in this case may be obtained by mixing the individual polymer solutions or by co-dissolving a polymer mixture. The aliphatic-aromatic polyester or the mixture thereof with the at least one additional polymer (and optionally the at least one further polymer) is the wall material of the subsequent microparticle. The wall material of the microparticle preferably has a solubility in dichloromethane at 25° C. and 1 bar of at least 50 g/1.

In this polyester solution, water or an aqueous solution of the pore former is emulsified in method step a). The resulting emulsion in this case is also referred to below as a w/o emulsion (water-in-oil emulsion).

The aqueous solution of the pore former is preferably a 0.1% to 10% by weight aqueous solution of the pore former, especially of a pore former selected from ammonium hydrogencarbonate and ammonium carbonate. Particular preference is given to ammonium carbonate, especially a 0.1 to 1 wt % solution of ammonium carbonate in water.

0.1 to 10 parts by weight of the pore former, based on the sum total of the polymers that form the wall material, are used. The polymers forming the wall material consist of at least one aliphatic-aromatic polyester, at least one additional polymer and optionally at least one further polymer. Preference is given to using 1 to 5 parts by weight, especially 1.3 to 3 parts by weight, of the pore former based on the sum total of the polymers that form the wall material.

The emulsification in method step a) is carried out using a disperser (rotor-stator or rotor-rotor). For example, homogenizers or dispersing machines having a high shear energy are suitable for preparing the w/o emulsion. The mean droplet size [D4,3] of the emulsion droplets is 0.2 to 30 μm.

The w/o emulsion produced in method step a) can optionally be stabilized with at least one dispersant. Dispersants suitable for w/o emulsions are generally known and are mentioned, for example, in EP 2794085 and in EP 3 007 815, the teaching of which is hereby expressly incorporated by reference.

To prepare the w/o emulsion in step a) and for stabilization thereof, instead of or together with the aforementioned dispersants, one or more emulsifiers can be used preferably having an HLB value according to Griffin in the range of 2 to 10, especially in the range of 3 to 8. The HLB value (HLB=hydrophilic lipophilic balance) according to Griffin (W. C. Griffin: Classification of surface-active agents by HLB. In: J. Soc. Cosmet. Chem. 1, 1949, pp. 311-326) is a dimensionless number between 0 and 20 which provides information on the water and oil solubility of a compound. Preferably, these are non-ionic emulsifiers having an HLB value according to Griffin in the range of 2 to 10, particularly in the range of 3 to 8. However, also suitable are anionic and zwitterionic emulsifiers having an HLB value according to Griffin in the range of 2 to 10, particularly in the range of 3 to 8.

Such emulsifiers are generally used in an amount from 0.1 to 10% by weight, especially 0.5 to 5% by weight, based on the total weight of the emulsion prepared in step a). In general, the emulsifier or emulsifiers are added to the solution of the polymer or polymers in the water-immiscible solvent before emulsifying water or the aqueous solution of the pore former into this solution.

Examples of suitable emulsifiers having an HLB value according to Griffin in the range of 2 to 10 are:
sorbitan fatty acid esters, particularly sorbitan mono-, di- and trifatty acid esters and mixtures thereof, such as sorbitan monostearate, sorbitan monooleate, sorbitan monolaurate, sorbitant tristearate, sorbitan sesquioleate, sorbitan dioleate, sorbitan trioleate;
fatty acid esters of glycerol or of polyglycerol such as glycero monostearate, glycerol distearate, glycerol monooleate, glycerol dioleate, glycerol monostearate monoacetate, glycerol monoacetate monooleate, polyglycerol polyrinoleate (E476), e.g. the commercially available emulsifier PGPR 90
lactyl esters of fatty acid monoesters of glycerol;
lecithins;
ethoxylated castor oils, ethoxylated hydrogenated castor oils with degrees of ethoxylation in the range of 2 to 20
ethoxylated and/or propoxylated C₁₂-C₂₂-alkanols having degrees of alkoxylation in the range of 2 to 10 e.g. stearyl alcohol ethoxylate having a degree of ethoxylation in the range of 2 to 5, stearyl alcohol ethoxylate-co-propoxylate having degrees of alkoxylation in the range of 2 to 8, isotridecyl ethoxylates having degrees of ethoxylation in the range of 2 to 3 and isotridecyl ethoxylate-co-propoxylates with degrees of alkoxylation in the range of 2 to 5,
ethoxylated and/or propoxylated C₄-C₁₆-alkylphenols having degrees of alkoxylation in the range of 2 to 10, e.g. nonylphenol ethoxylate having degrees of ethoxylation in the range of 2 to 5 and octylphenol ethoxylate having degrees of ethoxylation in the range of 2 to 5.

### Method Step b)

The emulsifying of the w/o emulsion in water to give the w/o/w emulsion (water-in-oil-in-water method emulsion) in method step b) is effected by stirring or shearing in the presence of at least one dispersant. It is possible here to meter an aqueous solution of the dispersant into the w/o emulsion. The dispersant is preferably initially charged in the form of an aqueous solution and the w/o emulsion is metered in. Depending on the energy input, it is possible to control the droplet size. Furthermore, the dispersant described below influences the size of the emulsion droplets in equilibrium.

The concentration of the dispersant in the aqueous dispersant solution is typically in the range of 0.1 to 8.0% by weight, particularly in the range of 0.3 to 5.0% by weight, and especially in the range of 0.5 to 4.0% by weight, based on the total weight of the aqueous solution.

The ratio by weight of the w/o emulsion provided in step a) to water, preferably in the form of the aqueous dispersant solution is typically in the range from 15:85 to 55:45, particularly in the range from 25:75 to 50:50, and especially in the range from 30:70 to 45:55.

In step b), the amount of dispersant used is typically at least 0.1% by weight, especially at least 0.2% by weight, based on the total weight of the w/o/w emulsion, and is particularly in the range of 0.1 to 2% by weight and especially in the range of 0.2 to 1% by weight, based on the total weight of the w/o/w emulsion.

Larger droplets with a mean droplet size of 100 to 600 μm are obtained with customary stirrers.

Suitable stirrer types include, e.g. propeller stirrers, impeller stirrers, disk stirrers, vane stirrers, anchor stirrers, pitched-blade stirrers, cross-beam stirrers, helical stirrers, and screw stirrers. It is possible in this case to input sufficient shearing energy by vigorous stirring to achieve droplet sizes of 10 to <100 μm, preferably to 50 μm.

Should even higher shear energy input be intended, it may be advantageous to use apparatus for generating a shear field.

The shear energy introduced can be directly derived from the power consumption of the apparatus for generating a shear field, taking account of the heat loss. Thus, the shear energy input into the w/o/w emulsion is preferably 250 to 25 000 watts·h/m³ batch size. Particular preference is given to an energy input of 500 to 15 000, especially 800 to 10 000, watts·h/m³ batch size, calculated based on the motor current.

Suitable apparatus for generating a shear field are comminuters operating according to the rotor-stator principle, such as toothed ring dispersing machines, colloid and corundum disk mills, and also high-pressure and ultrasound homogenizers. Preference is given to the use of the toothed ring dispersing machines operating by the rotor-stator principle for generating the shear field. The diameter of the rotors and stators is customarily in the range between 2 cm and 40 cm, depending on machine size and dispersing performance. The speed of rotation of such dispersing machines is generally in the range from 500 to 20 000 rpm, depending on the construction type. Of course, machines with large rotor diameters rotate at the lower end of the rotation speed range, while machines with small rotor diameters are usually operated at the upper end of the rotation speed range. The distance of the rotating parts from the stationary parts of the dispersing tool is generally 0.1 to 3 mm.

In a preferred embodiment, the final size of the emulsion droplets of the w/o/w emulsion should be an average diameter D[4,3] (determined by means of light scattering) of 100 to 600 μm. This final size is generally achieved just by stirring.

In a likewise preferred embodiment, the final size of the emulsion droplets of the w/o/w emulsion should have an average diameter of 10 to 100 μm, preferably 10 to 30 μm. This final size is typically achieved by means of shearing.

The w/o/w emulsion is produced in the presence of at least one dispersant. In one embodiment, the w/o/w emulsion can be produced in the presence of a mixture of different dispersants. Likewise, only one dispersant may also be used. Suitable dispersants are, for example, cellulose derivatives such as hydroxyethyl cellulose, methyl hydroxyethyl cellulose, methyl cellulose and carboxymethyl cellulose, polyvinylpyrrolidone, copolymers of vinylpyrrolidone, gelatin, gum arabic, xanthan, casein, polyethylene glycols, and partly hydrolyzed polyvinyl acetates (polyvinyl alcohols), and also methyl hydroxypropyl cellulose and mixtures of the aforementioned. Preferred dispersants are partially or fully hydrolyzed polyvinyl acetates (polyvinyl alcohols) and also methyl hydroxy(C₁-C₄)alkyl celluloses. Particular preference is given to partially hydrolyzed polyvinyl acetates, which are also referred to as partially hydrolyzed polyvinyl alcohols (PVA), preferably those having a degree of hydrolysis of 79% to 99.9%. In addition, PVA copolymers, as described in WO 2015/165836, are also suitable.

Methyl hydroxy(C₁-C₄)alkyl celluloses are understood to mean methyl hydroxy(C₁-C₄)alkyl celluloses of a wide variety of degrees of methylation and also degrees of alkoxylation. The preferred methyl hydroxy(C₁-C₄)alkyl celluloses have an average degree of substitution DS of 1.1 to 2.5 and a molar degree of substitution MS of 0.03 to 0.9.

Suitable methyl hydroxy(C₁-C₄)alkyl celluloses are for example methyl hydroxyethyl cellulose or methyl hydroxypropyl cellulose. A particularly preferred dispersant is methyl hydroxypropyl cellulose. Especially preferred dispersants are polyvinyl alcohols (PVAs), particularly polyvinyl alcohols having a degree of hydrolysis of 79% to 99.9%. A specific dispersant for step b) is a carboxy-modified anionic PVA having a proportion of carboxyl groups of 1 to 6 mol % and a degree of hydrolysis of 85 to 90 mol %, and especially preferably such a carboxy-modified anionic PVA of which a 4% by weight aqueous solution at 20° C. has a viscosity of 20.0 to 30.0 mPa·s.

In order to stabilize the w/o/w emulsion, the dispersant is particularly added to the aqueous phase. The concentration of the dispersant in the aqueous phase is typically in the range of 0.1 to 8.0% by weight, particularly in the range of 0.3 to 5.0% by weight, and especially in the range of 0.5 to 4.0% by weight, based on the total weight of the aqueous phase. The ratio by weight of the w/o emulsion provided in step a) to the aqueous phase comprising the dispersant is typically in the range from 15:85 to 55:45, particularly in the range from 25:75 to 50:50, and especially in the range from 30:70 to 45:55.

According to a preferred embodiment, carboxy-modified anionic PVA (having a degree of hydrolysis of 85 to 90 mol % and a viscosity of 20.0 to 30.0 mPa*s and a proportion of carboxyl groups of 1 to 6 mol %) is used as a 0.1% to 8% by weight aqueous solution, particularly as a 0.1 to 5% by weight aqueous solution and especially as a 0.3 to 4.0% by weight aqueous solution. Particular preference is given to aqueous solutions having a PVA content of 0.3% to 4% by weight, Likewise, it is possible to use aqueous solutions having a PVA content of 0.3 to 2.5% by weight, particularly solutions having a PVA content of 0.5 to 1.5% by weight.

According to a preferred method variant, in method step b) the emulsification to give the w/o/w emulsion is carried out with a stirrer at a stirring speed of 5000 to 15 000 rpm over a period of 1-30 minutes. The droplets produced thereby have a mean diameter of 0.2 to 30 μm.

According to a further preferred method variant, the emulsion is prepared at a stirring speed of 100-1000 rpm over a period of 1-30 minutes. The mean diameter of the droplets produced thereby is 100 to 600 μm.

During the emulsification, and optionally thereafter, the mixture is kept at a temperature in the range from 20 to 80° C. The temperature of the mixture is preferably selected such that it is below the glass transition temperature of the lowest softening amorphous polymer or below the melting point of the lowest melting crystalline polymer of the composition that forms the wall material. Higher temperatures are possible, but they may lead to partial closure of the pores over too long a period. The mixture is preferably kept at a temperature in the range from 20 to 45° C., especially from 20 to <40° C. Optionally, a vacuum may additionally be applied. For instance, it may be operated in the range of 600 to 800 mbar or below 200 mbar.

These measures, both the stirring/shearing and the temperature, and optionally the vacuum applied, lead to the water-immiscible solvent of the at least one aliphatic-aromatic polyester evaporating and the microparticles being left behind.

Provided that it is a solvent having a vapor pressure ≥450 hPa at 20° C., it is sufficient to stir the w/o/w emulsion obtained in b) at room temperature, 20° C. Depending on the amount of the solvent and the ambient temperature, such an operation lasts for a few hours. Depending on the solvent, it is possible to remove the solvent by raising the temperature to a temperature of up to 80° C. and/or by applying a slight vacuum.

For example, with solvents such as dichloromethane, according to a preferred embodiment the following is selected: 10 hours stirring at room temperature with 100 l/hour of nitrogen flow in a 2 l vessel, or 3 hours stirring at 45° C. jacket temperature with 100 l/hour of nitrogen flow in a 2 l vessel.

With solvents such as ethyl acetate, according to a further preferred embodiment the following is selected: 6 hours stirring at 60° C. with 100 l/hour of nitrogen flow.

In the course of the removal of the water-immiscible solvent, pore formation is observed in the walls of the microparticles.

The microparticles formed by removal of the water-immiscible solvent are removed in method step c) and preferably dried. “Dried” is understood to mean that the microparticles comprise a residual amount of water of ≤5% by weight, preferably ≤1% by weight, based on the microparticles. The drying may for example be carried out in a stream of air and/or by applying a vacuum, optionally in each case with heating. This is accomplished, depending on the size of the capsules, by means of convective driers such as spray driers, fluidized bed and cyclone driers, contact driers such as pan driers, paddle driers, contact belt driers, vacuum drying cabinet or radiative driers such as infrared rotary tube driers and microwave mixing driers.

The spherical microparticles obtained in this way are also a subject of the present invention. They are characterized in that they are easy to fill, in that they are for example suspended in a solution.

The inventive composition consists of spherical microparticles constructed of wall material and at least one cavity, and having pores at their surface.

According to a preferred embodiment, the inventive spherical microparticles having a particle size in the range from 100 to 600 μm have a bulk density (determined according to DIN EN ISO 60: 1999) of 0.1 to 0.5 g/cm³, preferably 0.15-0.4 g/cm³, especially of 0.15 to 0.3 g/cm³.

The inventive spherical microparticles are used as carrier substance for filling with an aroma chemical, preferably a fragrance, preferably in a solvent or diluent.

An “aroma chemical” is a generic term for compounds which may be used as “fragrance” and/or as “flavoring”.

In the context of the present invention, “fragrance” is understood to mean natural or synthetic substances having intrinsic odor.

In the context of the present invention, “flavoring” is understood to mean natural or synthetic substances having intrinsic flavor.

In the context of the present invention, “odor” or “olfactory perception” is the interpretation of the sensory stimuli which are sent from the chemoreceptors in the nose or other olfactory organs to the brain of a living being. The odor can be a result of sensory perception of the nose of fragrances, which occurs during inhalation. In this case, the air serves as odor carrier.

In the context of the present invention, a “perfume” is a mixture of fragrances and carriers such as, in particular, an alcohol.

In the context of the present invention, a “perfume composition” is a perfume comprising different amounts of individual components harmoniously balanced with one another. The properties of the individual constituents are employed in order to achieve a new overall image in the combination, wherein the characteristics of the ingredients retire into the background but without being suppressed.

In the context of the present invention, a “perfume oil” is a concentrated mixture of several fragrances which are employed, for example, in alcoholic solutions, for perfuming different products.

In the context of the present invention, a “solvent for fragrances” serves as the diluent of the fragrances to be used according to the invention or the fragrance composition according to the invention but without having any intrinsic odorous properties. Some solvents also have fixing properties.

The fragrance, or a mixture of several fragrances, may be admixed to 0.1 to 99 wt % with a diluent or solvent. Preference is given to at least 40 wt % solutions, more preferably at least 50 wt % solutions, further preferably at least 60 wt % solutions, more preferably at least 70 wt % solutions, particularly preferably at least 80 wt % solutions, especially preferably at least 90 wt % solutions, preferably in olfactorily acceptable solutions.

Examples of preferred olfactorily acceptable solvents are ethanol, isopropanol, dipropylene glycol (DPG), 1,2-propylene glycol, 1,2-butylene glycol, glycerol, diethylene glycol monoethyl ether, diethyl phthalate (DEP), 1,2-cyclohexane dicarboxylic acid diisononyl ester, isopropyl myristate (IPM), triethyl citrate (TEC), benzyl benzoate (BB) and benzyl acetate. In this case, preference is given in turn to ethanol, diethyl phthalate, propylene glycol, dipropylene glycol, triethyl citrate, benzyl benzoate and isopropyl myristate.

### Fragrances:

Microparticles filled with a fragrance in accordance with the invention comprise at least one fragrance, preferably 2, 3, 4, 5, 6, 7, 8 or more fragrances, which are for example selected from: alpha-hexylcinnamaldehyde, 2-phenoxyethyl isobutyrate (Phenirat¹), dihydromyrcenol (2,6-dimethyl-7-octen-2-ol), methyl dihydrojasmonate (preferably having a cis-isomer content of more than 60 wt %) (Hedione⁹, Hedione HC⁹), 4,6,6,7,8,8-hexamethyl-1,3,4,6,7,8-hexahydrocyclopenta[g]benzopyran (Galaxolide³), tetrahydrolinalool (3,7-dimethyloctan-3-ol), ethyl linalool, benzyl salicylate, 2-methyl-3-(4-tert-butylphenyl)propanal (Lilial²), cinnamyl alcohol, 4,7-methano-3a,4,5,6,7,7a-hexahydro-5-indenyl acetate and/or 4,7-methano-3a,4,5,6,7,7a-hexahydro-6-indenyl acetate (Herbaflorat¹), citronellol, citronellyl acetate, tetrahydrogeraniol, vanillin, linalyl acetate, styralyl acetate (1-phenylethyl acetate), octahydro-2,3,8,8-tetramethyl-2-acetonaphthone and/or 2-acetyl-1,2,3,4,6,7,8-octahydro-2,3,8,8-tetramethylnaphthalene (Iso E Super³), hexyl salicylate, 4-tert-butylcyclohexyl acetate (Oryclone¹), 2-tert-butylcyclohexyl acetate (Agrumex HC¹), alpha-ionone (4-(2,2,6-trimethyl-2-cyclohexen-1-yl)-3-buten-2-one), n-alpha-methylionone, alpha-isomethylionone, coumarin, terpinyl acetate, 2-phenylethyl alcohol, 4-(4-hydroxy-4-methylpentyl)-3-cyclohexenecarboxaldehyde (Lyral³), alpha-amylcinnamaldehyde, ethylene brassylate, (E)- and/or (Z)-3-methylcyclopentadec-5-enone (Muscenone⁹), 15-pentadec-11-enolide and/or 15-pentadec-12-enolide (Globalide¹), 15-cyclopentadecanolide (Macrolide¹), 1-(5,6,7,8-tetrahydro-3,5,5,6,8,8-hexamethyl-2-naphthalenyl)ethanone (Tonalide¹⁰), 2-isobutyl-4-methyltetrahydro-2H-pyran-4-ol (Florol⁹), 2-ethyl-4-(2,2,3-trimethyl-3-cyclopenten-1-yl)-2-buten-1-ol (Sandolene¹), cis-3-hexenyl acetate, trans-3-hexenylacetate, trans-2-cis-6-nonadienol, 2,4-dimethyl-3-cyclohexenecarboxaldehyde (Vertocitral¹), 2,4,4,7-tetramethyl-oct-6-en-3-one (Claritone¹), 2,6-dimethyl-5-hepten-1-a1 (Melonal²), borneol, 3-(3-isopropylphenyl)butanal (Florhydral²), 2-methyl-3-(3,4-methylenedioxyphenyl)propanal(Helional³),3-(4-ethylphenyl)-2,2-dimethylpropanal(Florazon¹), 7-methyl-2H-1,5-benzodioxepin-3(4H)-one (Calone⁹⁵¹⁵), 3,3,5-trimethylcyclohexyl acetate (preferably with a content of cis-isomers of 70 wt %) or more and 2,5,5-trimethyl-1,2,3,4,4a,5,6,7-octahydronaphthalen-2-ol (Ambrinol S¹). In the context of the present invention, the fragrances mentioned above are accordingly preferably combined with mixtures according to the invention.

If trade names are specified above, these refer to the following sources: ¹ trade name of Symrise GmbH, Germany;² trade name of Givaudan AG, Switzerland;³ trade name of International Flavors & Fragrances Inc., USA;⁵ trade name of Danisco Seillans S. A., France;⁹ trade name of Firmenich S. A., Switzerland;¹⁰ trade name of PFW Aroma Chemicals B. V., the Netherlands.

Further fragrances with which the (E/Z)-cyclopentadecenylcarbaldehydes (I)-(III) may be combined, for example, to give a fragrance composition are found, for example, in S. Arctander, Perfume and Flavor Chemicals, Vol. I and II, Montclair, N. J., 1969, Author's edition or K. Bauer, D. Garbe and H. Surburg, Common Fragrance and Flavor Materials, 4th. Ed., Wiley—VCH, Weinheim 2001. Specifically, the following may be mentioned:

extracts from natural raw materials such as essential oils, concretes, absolutes, resins, resinoids, balsams, tinctures, for example
ambra tincture; amyris oil; angelica seed oil; angelica root oil; anise oil; valerian oil; basil oil; tree moss absolute; bay oil; mugwort oil; benzoin resin; bergamot oil; beeswax absolute; birch tar oil; bitter almond oil; savory oil; bucco leaf oil; cabreuva oil; cade oil; calamus oil; camphor oil; cananga oil; cardamom oil; cascarilla oil; cassia oil; cassie absolute; castoreum absolute; cedar leaf oil; cedar wood oil; cistus oil; citronella oil; lemon oil; copaiba balsam; copaiba balsam oil; coriander oil; costus root oil; cumin oil; cypress oil; davana oil; dill oil; dill seed oil; eau de brouts absolute; oakmoss absolute; elemi oil; estragon oil; eucalyptus citriodora oil; eucalyptus oil; fennel oil; spruce needle oil; galbanum oil; galbanum resin; geranium oil; grapefruit oil; guaiac wood oil; gurjun balsam; gurjun balsam oil; helichrysum absolute; helichrysum oil; ginger oil; iris root absolute; iris root oil; jasmine absolute; calamus oil; camellia oil blue; camellia oil roman; carrot seed oil; cascarilla oil; pine needle oil; spearmint oil; cumin oil; labdanum oil; labdanum absolute; labdanum resin; lavandin absolute; lavandin oil; lavender absolute; lavender oil; lemon grass oil; lovage oil; lime oil distilled; lime oil pressed; linalool oil; litsea cubeba oil; laurel leaf oil; macis oil; marjoram oil; mandarin oil; massoia bark oil; mimosa absolute; musk seed oil; musk tincture; clary sage oil; nutmeg oil; myrrh absolute; myrrh oil; myrtle oil; clove leaf oil; clove flower oil; neroli oil; olibanum absolute; olibanum oil; opopanax oil; orange blossom absolute; orange oil; oregano oil; palmarosa oil; patchouli oil; perilla oil; Peruvian balsam oil; parsley leaf oil; parsley seed oil; petitgrain oil; peppermint oil; pepper oil; allspice oil; pine oil; poley oil; rose absolute; rosewood oil; rose oil; rosemary oil; sage oil dalmatian; sage oil Spanish; sandalwood oil; celery seed oil; spike lavender oil; star anise oil; styrax oil; tagetes oil; fir needle oil; tea tree oil; turpentine oil; thyme oil; tolu balsam; tonka absolute; tuberose absolute; vanilla extract; violet leaf absolute; verbena oil; vetiver oil; juniper berry oil; wine yeast oil; vermouth oil; wintergreen oil; ylang oil; hyssop oil; civet absolute; cinnamon leaf oil; cinnamon bark oil; and fractions thereof or ingredients isolated therefrom;
individual fragrances from the group of hydrocarbons, such as e.g. 3-carene; alpha-pinene; beta-pinene; alpha-terpinene; gamma-terpinene; p-cymene; bisabolene; camphene; caryophyllene; cedrene; farnesene; limonene; longifolene; myrcene; ocimene; valencene; (E,Z)-1,3,5-undecatriene; styrene; diphenylmethane;
the aliphatic alcohols such as e.g. hexanol; octanol; 3-octanol; 2,6-dimethylheptanol; 2-methyl-2-heptanol; 2-methyl-2-octanol; (E)-2-hexenol; (E)- and (Z)-3-hexenol; 1-octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;
the aliphatic aldehydes and acetals thereof such as e.g. hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-9-undecenal; 2,6,10-trimethyl-5,9-undecadienal; heptanal diethylacetal; 1,1-dimethoxy-2,2,5-trimethyl-4-hexene; citronellyloxyacetaldehyde; (E/Z)-1-(1-methoxypropoxy)-3-hexene; the aliphatic ketones and oximes thereof such as e.g. 2-heptanone; 2-octanone; 3-octanone; 2-nonanone; 5-methyl-3-heptanone; 5-methyl-3-heptanone oxime; 2,4,4,7-tetramethyl-6-octen-3-one; 6-methyl-5-hepten-2-one;
the aliphatic sulfur-containing compounds such as e.g. 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;
the aliphatic nitriles such as e.g. 2-nonenenitrile; 2-undecenenitrile; 2-tridecenenitrile; 3,12-tridecadienenitrile; 3,7-dimethyl-2,6-octadienenitrile; 3,7-dimethyl-6-octenenitrile;
the esters of aliphatic carboxylic acids such as e.g. (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; (E/Z)-ethyl-2,4-decadienoate; methyl 2-octinate; methyl 2-noninate; allyl 2-isoamyloxy acetate; methyl-3,7-dimethyl-2,6-octadienoate; 4-methyl-2-pentyl crotonate; the acyclic terpene alcohols such as e.g. geraniol; nerol; linalool; lavandulol; nerolidol; farnesol; tetrahydrolinalool; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and the formates, acetates, propionates, isobutyrates, butyrates, isovalerates, pentanoates, hexanoates, crotonates, tiglinates and 3-methyl-2-butenoates thereof;
the acyclic terpene aldehydes and ketones such as e.g. geranial; neral; citronellal; 7-hydroxy-3,7-dimethyloctanal; 7-methoxy-3,7-dimethyloctanal; 2,6,10-trimethyl-9-undecenal; geranyl acetone; as well as the dimethyl and diethyl acetals of geranial, neral, 7-hydroxy-3,7-dimethyloctanal; the cyclic terpene alcohols such as e.g. menthol; isopulegol; alpha-terpineol; terpineol-4; menthan-8-ol; menthan-1-ol; menthan-7-ol; borneol; isoborneol; linalool oxide; nopol; cedrol; ambrinol; vetiverol; guajol; and the formates, acetates, propionates, isobutyrates, butyrates, isovalerates, pentanoates, hexanoates, crotonates, tiglinates and 3-methyl-2-butenoates thereof;
the cyclic terpene aldehydes and ketones such as e.g. menthone; isomenthone; 8-mercaptomenthan-3-one; carvone; camphor; fenchone; alpha-ionone; beta-ionone; alpha-nmethylionone; beta-n-methylionone; alpha-isomethylionone; beta-isomethylionone; alpha-irone; alpha-damascone; beta-damascone; beta-damascenone; delta-damascone; gammadamascone; 1-(2,4,4-trimethyl-2-cyclohexen-1-yl)-2-buten-1-one; 1,3,4,6,7,8a-hexahydro-1,1,5,5-tetramethyl-2H-2,4a-methanonaphthalene-8(5H)-one; 2-methyl-4-(2,6,6-trimethyl-1-cyclohexen-1-yl)-2-butenal; nootkatone; dihydronootkatone; 4,6,8-megastigmatrien-3-one; alpha-sinensal; beta-sinensal; acetylated cedar wood oil (methyl cedryl ketone);
the cyclic alcohols such as e.g. 4-tert-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;
the cycloaliphatic alcohols such as e.g. alpha-3,3-trimethylcyclohexylmethanol; 1-(4-isopropylcyclohexyl)ethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol; 3-methyl-5-(2,2,3-trimethyl-3-cyclopent-1-yl)pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;
the cyclic and cycloaliphatic ethers such as e.g. cineol; cedryl methyl ether; cyclododecyl methyl ether; 1,1-dimethoxycyclododecane; (ethoxymethoxy)cyclododecane; alpha-cedrene epoxide; 3a,6,6,9a tetramethyldodecahydronaphtho[2,1-b]furan; 3a-ethyl-6,6,9a-trimethyldodecahydronaphtho[2,1-b]furan; 1,5,9-trimethyl-13-oxabicyclo[10.1.0]trideca-4,8-diene; rose oxide; 2-(2,4-dimethyl-3-cyclohexen-1-yl)-5-methyl-5-(1-methylpropyl)-1,3-dioxane;
the cyclic and macrocyclic ketones such as e.g. 4-tert-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; cis-3-methylpent-2-en-1-yl-cyclopent-2-en-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert-pentylcyclohexanone; cyclohexadec-5-en-1-one; 6,7-dihydro-1,1,2,3,3-pentamethyl-4(5H)-indanone; 8 cyclohexadecen-1-one; 7-cyclohexadecen-1-one; (7/8)-cyclohexadecen-1-one; 9 cycloheptadecen-1-one; cyclopentadecanone; cyclohexadecanone;
the cycloaliphatic aldehydes such as e.g. 2,4-dimethyl-3-cyclohexenecarbaldehyde; 2-methyl-4-(2,2,6-trimethylcyclohexen-1-yl)-2-butenal; 4-(4-hydroxy-4-methylpentyl)-3-cyclohexenecarbaldehyde; 4-(4-methyl-3-penten-1-yl)-3-cyclohexenecarbaldehyde;
the cycloaliphatic ketones such as e.g. 1-(3,3-dimethylcyclohexyl)-4-penten-1-one; 2,2-dimethyl-1-(2,4-dimethyl-3-cyclohexen-1-yl)-1-propanone; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl 2,6,10-trimethyl-2,5,9-cyclododecatrienyl ketone; tert-butyl(2,4-dimethyl-3-cyclohexen-1-yl) ketone;
the esters of cyclic alcohols such as e.g. 2-tert-butylcyclohexyl acetate; 4-tert-butylcyclohexyl acetate; 2-tert-pentylcyclohexyl acetate; 4-tert-pentylcyclohexyl acetate; 3,3,5-trimethylcyclohexyl acetate; decahydro-2-naphthyl acetate; 2-cyclopentylcyclopentyl crotonate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5 or 6-indenyl acetate; 4,7-methano-3a,4,5,6,7,7a-hexahydro-5 or 6 indenyl propionate; 4,7-methano-3a,4,5,6,7,7a-hexahydro-5 or 6-indenyl isobutyrate; 4,7-methanooctahydro-5 or 6-indenyl acetate;
the esters of cycloaliphatic alcohols such as e.g. 1-cyclohexylethyl crotonate;
the esters of cycloaliphatic carboxylic acids such as e.g. allyl 3-cyclohexylpropionate; allyl cyclohexyloxyacetate; cis and trans-methyl dihydrojasmonate; cis and trans-methyl jasmonate; methyl 2-hexyl-3-oxocyclopentanecarboxylate; ethyl 2-ethyl-6,6-dimethyl-2-cyclohexenecarboxylate; ethyl 2,3,6,6-tetramethyl-2-cyclohexenecarboxylate; ethyl 2-methyl-1,3-dioxolane-2-acetate;
the araliphatic alcohols such as e.g. benzyl alcohol; 1-phenylethyl alcohol, 2-phenylethyl alcohol, 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;
the esters of araliphatic alcohols and aliphatic carboxylic acids such as e.g. benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha, alpha-dimethylphenylethyl acetate; alpha, alpha-dimethylphenylethyl butyrate; cinnamyl acetate; 2-phenoxyethyl isobutyrate; 4-methoxybenzyl acetate;
the araliphatic ethers such as e.g. 2-phenylethyl methyl ether; 2-phenylethyl isoamyl ether; 2-phenylethyl 1-ethoxyethyl ether; phenylacetaldehyde dimethyl acetal; phenylacetaldehyde diethyl acetal; hydratropaaldehyde dimethyl acetal; phenylacetaldehyde glycerol acetal; 2,4,6-trimethyl-4-phenyl-1,3-dioxane; 4,4a,5,9b-tetrahydroindeno[1,2-d]-m-dioxine; 4,4a,5,9b-tetrahydro-2,4-dimethylindeno[1,2-d]-m-dioxine;
the aromatic and araliphatic aldehydes such as e.g. benzaldehyde; phenylacetaldehyde; 3-phenylpropanal; hydratropaaldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-tert-butylphenyl)propanal; 2-methyl-3-(4-isobutylphenyl)propanal; 3-(4-tert-butylphenyl)propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxybenzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal;
the aromatic and araliphatic ketones such as e.g. acetophenone; 4-methylacetophenone; 4-methoxyacetophenone; 4-tert-butyl-2,6-dimethylacetophenone; 4-phenyl-2-butanone; 4-(4-hydroxyphenyl)-2-butanone; 1-(2-naphthalenyl)ethanone; 2-benzofuranylethanone; (3-methyl-2-benzofuranyl)ethanone; benzophenone; 1,1,2,3,3,6-hexamethyl-5-indanyl methyl ketone; 6-tertbutyl-1,1-dimethyl-4-indanyl methyl ketone; 1-[2,3-dihydro-1,1,2,6-tetramethyl-3-(1-methylethyl)-1 H-5-indenyl]ethanone; 5′,6′,7′,8′-tetrahydro-3′,5′,5′,6′,8′,8′-hexamethyl-2-acetonaphthone;
the aromatic and araliphatic carboxylic acids and esters thereof such as e.g. benzoic acid; phenylacetic acid; methyl benzoate; ethyl benzoate; hexyl benzoate; benzyl benzoate; methyl phenylacetate; ethyl phenylacetate; geranyl phenylacetate; phenylethyl phenylacetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;
the nitrogen-containing aromatic compounds such as e.g. 2,4,6-trinitro-1,3-dimethyl-5-tert-butylbenzene; 3,5-dinitro-2,6-dimethyl-4-tert-butylacetophenone; cinnamonitrile; 3-methyl-5-phenyl-2-pentenonitrile; 3-methyl-5-phenylpentanonitrile; methyl anthranilate; methyl N-methylanthranilate; Schiff's bases of methyl anthranilate with 7-hydroxy-3,7-dimethyloctanal, 2-methyl-3-(4-tert-butylphenyl)propanal or 2,4-dimethyl-3-cyclohexenecarbaldehyde; 6-isopropylquinoline; 6-isobutylquinoline; 6-sec-butylquinoline; 2-(3-phenylpropyl)pyridine; indole; skatole; 2-methoxy-3-isopropylpyrazine; 2-isobutyl-3-methoxypyrazine;
the phenols, phenyl ethers and phenyl esters such as e.g. estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresyl phenylacetate;
the heterocyclic compounds such as e.g. 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;
the lactones such as e.g. 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 4-methyl-1,4-decanolide; 1,15-pentadecanolide; cis and trans-11-pentadecen-1,15-olide; cis and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin.

Furthermore, compounds as described in PCT/EP2015/072544 are suitable as fragrances.

Particular preference is given to mixtures of L-menthol and/or DL-menthol, L-menthone, L-menthyl acetate, which are highly sought-after as analogs or substitutes for what are referred to as synthetic dementholized oils (DMOs). The mixtures of these minty compositions are preferably used in the ratio L-menthol or DL-menthol 20-40 wt %, L-menthone 20-40% and L-menthyl acetate 0-20%.

The present invention further relates to a method for filling and optionally closing the filled microparticles.

The spherical microparticles are filled by impregnating the spherical microparticles with at least one aroma chemical, preferably a fragrance.

The spherical microparticles impregnated with at least one aroma chemical are referred to as aroma chemical preparation.

The term “impregnating” includes any bringing of the microparticles into contact with at least one aroma chemical which results in the cavity present in the unfilled microparticles being filled at least partly by the aroma chemical(s) or some of the gas present in the microparticles being displaced by the liquid. In particular, the term “impregnating” includes the bringing of the microparticles into contact with the at least one aroma chemical which results in the cavity present in the unfilled microparticles being filled to an extent of at least 50%, especially to an extent of at least 70%, or being completely filled, or the majority of the gas present in the microparticles being displaced by the liquid.

The impregnating can take place with a liquid aroma chemical or with a solution of at least one aroma chemical.

In one embodiment of the invention, the microparticles are impregnated by suspending the microparticles in a liquid aroma chemical or in a solution of at least one aroma chemical.

In one embodiment of the invention, the microparticles are impregnated using a method in which the aroma chemical is present in finely divided form, preferably in the form of droplets. In particular, to impregnate the unladen microparticles, a liquid aroma chemical or a solution of at least one aroma chemical can be applied in finely divided form, especially in the form of droplets, to the unladen microparticles. For this purpose, the microparticles are naturally used in solid form, particularly in the form of a powder. In particular, the unladen microparticles as a powder can be subjected to spray application or dropwise application with the respective liquid comprising the aroma chemical. Surprisingly, the liquid droplets are rapidly absorbed by the unladen microparticles. Also in this manner, the liquid used for the impregnation and thus the aroma chemical can be precisely metered in such that removal of excess liquid can be avoided, or the inconvenience associated therewith can be reduced.

In general, for this purpose, the unladen microparticles in solid form, particularly in the form of a powder, will be initially charged in a mixer for the mixing of solids with liquids and the liquid comprising the at least one aroma chemical is added, preferably in finely divided form, especially in the form of droplets, for example in the form of discrete droplets or as a spray mist. In particular, the respective liquid comprising the at least one aroma chemical is applied in finely divided form, especially in the form of droplets, to the microparticles to be loaded while in motion. For example, it is possible in a suitable manner to move the microparticles to be laden, in particular to create a fluidized bed of the microparticles to be laden or fluidized layer of microparticles to be laden, and to apply, for example by spraying or dropwise, the respective liquid in finely divided form to the agitated microparticles or microparticles present in the fluidized bed or fluidized layer. The spray application or droplet application can be effected in a known manner by means of one or more nozzles, e.g. by means of one-phase or two-phase nozzles or by means of droppers. Suitable mixing apparatuses are dynamic mixers, especially forced mixers, or those with a mixer shaft, e.g. shovel mixers, paddle mixers or ploughshare mixers, but also free-fall mixers of this kind, e.g. drum mixers, and fluidized bed mixers. The duration of the mixing operation depends on the type of mixer and the viscosity of the liquid comprising the aroma chemical at loading temperature and hence on the diffusion rate of the liquid into the microparticles. The time required for loading can be determined in a simple manner by the person skilled in the art. It is generally 1 minute to 5 hours, particularly 2 minutes to 2 hours or 5 minutes to 1 hour. Preferably, the respective liquid comprising the at least one aroma chemical is used in an amount of 0.2 to 5 parts by weight, preferably 0.5 to 4 parts by weight, based on 1 part by weight of the unladen microparticles. The spray application or dropwise application is generally at a temperature in the range from 0 to 80° C., particularly in the range from 10 to 70° C. and especially in the range from 20 to 60° C.

### Suspending

In one embodiment, the spherical microparticles are filled by the spherical microparticles being suspended in a liquid aroma chemical or solution of an aroma chemical, preferably a fragrance. In order to prepare the suspension, for example magnetic stirrers, rollers, shakers, or various wall-adjacent stirring members (e.g. anchor stirrer, helical stirrer) are suitable. The duration of the mixing procedure is dependent on the solution of the aroma chemical and is generally from 5 minutes to 12 hours.

The suspending is for example carried out over a period of several hours, preferably for longer than 1 hour, for example 5 hours, by mixing at room temperature. Longer suspending is possible but after a certain point no further increase of the loading will occur.

In one embodiment, the spherical microparticles are filled by
e) the spherical microparticles being suspended in a liquid aroma chemical or a solution of at least one aroma chemical, and
f) subsequently, the microparticles obtained after e) being kept at a temperature in the range from 35 to 200° C. over a period of 1 minute to 10 hours, preferably at a temperature in the range from 40 to 140° C., preferably from 45 to 80° C., over a period of 1 hour to 10 hours, and
g) optionally the spherical microparticles subsequently being removed.

Preferably, 1 part by weight of spherical microparticles is suspended in 0.2 to 5 parts by weight, preferably 0.5 to 4 parts by weight, preferably 1 to 3 parts by weight, of the aroma chemical or the solution thereof.

The suspension obtained after e) is generally kept at a temperature in the range from 35 to 200° C. for 1 minute to 10 hours. The suspension is preferably kept at a temperature in the range from 40 to 140° C., especially from 45 to 80° C. for 1 hour to 10 hours. In this step, the majority of the pores, preferably all pores of the microparticles are sealed. By means of the selection of temperature and time, the extent of sealing of the pores can be controlled.

According to a preferred embodiment, spherical microparticles consisting of a polymer material made of 30 to 70% by weight PBAT and 30 to 70% by weight polycaprolactone are selected. These microparticles are mixed for at least 1 hour with at least one liquid aroma chemical or a solution of at least one aroma chemical, and subsequently heated to a temperature in the range from 55 to 70° C. and stirred at this temperature for at least 3 hours.

Preference is given to spherical microparticles consisting of a polymer material made of 55% by weight PBAT and 45% by weight polycaprolactone. After filling, these microparticles are heated to a temperature of 60° C. and stirred at this temperature for 5 hours. Thereafter, the suspension is cooled to room temperature and the filled microparticles are removed.

According to a preferred embodiment, spherical microparticles consisting of a polymer material made of 30 to 70% by weight PBSeT and 30 to 70% by weight polycaprolactone are selected. These microparticles are mixed for at least 1 hour with at least one liquid aroma chemical or a solution of at least one aroma chemical, and subsequently heated to a temperature in the range from 55 to 70° C. and stirred at this temperature for at least 3 hours.

Preference is given to spherical microparticles consisting of a polymer material made of 55% by weight PBSeT and 45% by weight polycaprolactone. After filling, these microparticles are heated to a temperature of 60° C. and stirred at this temperature for 5 hours. Thereafter, the suspension is cooled to room temperature and the filled microparticles are removed.

It is assumed that the filled microparticles are closed by coalescence of the pores, by the suspension, depending on the polymer of the microparticle that forms the wall material, being heated to above its melting point or to above its glass transition temperature when it does not have a melting point. If the wall material is a composition of at least two polymers, the same principle applies wherein in that case the values of both polymers are taken into consideration.

Furthermore, the present invention relates to a method for preparing an aroma chemical preparation, in which the spherical microparticles obtained according to the method are suspended in an aroma chemical or in a solution of at least one aroma chemical, and are subsequently kept at a temperature in the range from 35 to 200° C., preferably from 40 to 140° C., particularly from 45 to 80° C., for a period from 1 minute to 10 hours.

According to a preferred embodiment, spherical microparticles are impregnated with an aroma chemical, wherein the spherical microparticles are selected from spherical microparticles consisting of a polymer material made of 30 to 70% by weight PBSeT and 30 to 70% by weight polycaprolactone and spherical microparticles consisting of a polymer material made of 30 to 70% by weight PBAT and 30 to 70% by weightpolycaprolactone.

Particular preference is given to spherical microparticles consisting of a polymer material made of 55% by weight PBAT and 45% by weight polycaprolactone and spherical microparticles consisting of a polymer material made of 55% by weight PBSeT and 45% by weight polycaprolactone.

The present application relates to the spherical microparticles obtained by this method and also to the use of the filled microparticles obtained by filling and optionally sealing, in agents selected from perfumes, washing and cleaning agents, cosmetic agents, body care agents, hygiene articles, food, food supplements, scent dispensers and fragrances.

Furthermore, it relates to the use of the spherical microparticles or the aroma chemical preparation, wherein it is used in an agent selected from perfumes, washing and cleaning agents, cosmetic agents, body care agents, hygiene articles, food, food supplements, scent dispensers or fragrances.

The filled spherical microparticles according to the invention are suitable for the controlled release of aroma chemicals.

Optionally, the filled and optionally closed microparticles are removed from the solution of aroma chemical that was added in excess. The methods suitable therefor are, e.g. filtration, centrifugation, decanting, vacuum distillation and spray drying.

It may be advantageous to remove any residual water present on the microparticles. This can be effected, for example, by rinsing with ethanol or acetone, and/or blowing the microparticles dry with an inert gas such as air, nitrogen or argon. Optionally, for this purpose, predried and/or preheated inert gases may also be used. The filled microparticles are preferably subsequently rinsed, preferably with aqueous propanediol solution, for example as 10 wt % solution.

Commonly known drying methods may be used for drying. For example, the particles may be dried by means of convective dryers such as spray dryers, fluidized bed, cyclone dryers, contact dryers such as pan dryers, paddle dryers, contact belt dryers, vacuum drying cabinet or radiative dryers such as infrared rotary tube dryer and microwave mixing dryer.

The inventive spherical microparticles filled with at least one aroma chemical or the solution of at least one aroma chemical, preferably a fragrance or a solution of a fragrance, may be incorporated into a variety of products or applied to such products. Such agents comprise the spherical microparticles or an aroma chemical preparation preferably in a proportion by weight of 0.01 to 99.9 wt % based on the total weight of the composition.

Spherical microparticles and the aroma chemical preparations according to the invention can be used in the production of perfumed articles. The olfactory properties and also the physical properties and the non-toxicity of the inventive microparticles highlight their particular suitability for the intended uses mentioned.

The use of the microparticles proves to be particularly advantageous in conjunction with top notes of compositions, for example in perfume compositions comprising dihydrorosan, rose oxide or other readily volatile fragrances, e.g. iso-amyl acetate, prenyl acetate or methylheptenone. In this case, the release of the important, sought-after top notes is effectively delayed. The fragrance or aroma compositions are accordingly metered in at the suitable point in the requisite amount. In the mint compositions of L-menthol, DL-menthol, L-menthone and L-menthyl acetate described, aside from the aroma effect, a cooling effect also is applied in a targeted manner, e.g. in chewing gums, confectionery, cosmetic products, and industrial applications such as in textiles or superabsorbents. A further advantage lies in the high material compatibility of the microparticles, even with reactive or unstable components such as aldehydes, esters, pyrans/ethers, which may exhibit secondary reactions on the surfaces.

The positive properties contribute to use of the aroma chemical preparations according to the invention with particular preference in perfume products, personal care products, hygiene articles, textile detergents and in cleaning products for solid surfaces.

The perfumed article is selected, for example, from perfume products, personal care products, hygiene articles, textile detergents and cleaning products for solid surfaces. Preferred perfumed articles of the invention are also selected from:

perfume products selected from perfume extracts, eau de parfums, eau de toilettes, eau de colognes, eau de solide, extrait parfum, air fresheners in liquid form, gel form or a form applied to a solid carrier, aerosol sprays, scented cleaners and scented oils;
personal care products selected from aftershaves, pre-shave products, splash colognes, solid and liquid soaps, shower gels, shampoos, shaving soaps, shaving foams, bath oils, cosmetic emulsions of the oil-in-water type, of the water-in-oil type and of the water-in-oil-in-water type, for example skin creams and lotions, face creams and lotions, sunscreen creams and lotions, aftersun creams and lotions, hand creams and lotions, foot creams and lotions, hair removal creams and lotions, aftershave creams and lotions, tanning creams and lotions, hair care products, for example hairsprays, hair gels, setting hair lotions, hair conditioners, hair shampoo, permanent and semipermanent hair colorants, hair shaping compositions such as cold waves and hair smoothing compositions, hair tonics, hair creams and hair lotions, deodorants and antiperspirants, for example underarm sprays, roll-ons, deodorant sticks, deodorant creams, products of decorative cosmetics, for example eye shadows, nail varnishes, make-ups, lipsticks, mascara, toothpaste, dental floss;
hygiene articles selected from candles, lamp oils, joss sticks, propellants, rust removers, perfumed freshening wipes, armpit pads, baby diapers, sanitary towels, toilet paper, cosmetic wipes, pocket tissues, dishwasher deodorizer;
cleaning products for solid surfaces selected from perfumed acidic, alkaline and neutral cleaners, for example floor cleaners, window cleaners, dishwashing detergents, bath and sanitary cleaners, scouring milk, solid and liquid toilet cleaners, powder and foam carpet cleaners, waxes and polishes such as furniture polishes, floor waxes, shoe creams, disinfectants, surface disinfectants and sanitary cleaners, brake cleaners, pipe cleaners, limescale removers, grill and oven cleaners, algae and moss removers, mold removers, facade cleaners;
textile detergents selected from liquid detergents, powder detergents, laundry pretreatments such as bleaches, soaking agents and stain removers, fabric softeners, washing soaps, washing tablets.

In a further aspect, the aroma chemical preparations according to the invention are suitable for use in surfactant-containing perfumed articles. This is because there is frequently a search—especially for the perfuming of surfactant-containing formulations, for example, cleaning products (in particular dishwashing compositions and all-purpose cleaners)—for fragrances and/or fragrance compositions with a rose topnote and marked naturalness.

In a further aspect, the aroma chemical preparations according to the invention can be used as products for providing (a) hair or (b) textile fibers with a rosy odor note.

The aroma chemical preparations to be used according to the invention are therefore particularly well suited for use in surfactant-containing perfumed articles.

It is preferred if the perfumed article is one of the following:
an acidic, alkaline or neutral cleaner which is selected in particular from the group consisting of all-purpose cleaners, floor cleaners, window cleaners, dishwashing detergents, bath and sanitary cleaners, scouring milk, solid and liquid toilet cleaners, powder and foam carpet cleaners, liquid detergents, powder detergents, laundry pretreatments such as bleaches, soaking agents and stain removers, fabric softeners, washing soaps, washing tablets, disinfectants, surface disinfectants,
an air freshener in liquid form, gel-like form or a form applied to a solid carrier or as an aerosol spray,
a wax or a polish, which is selected in particular from the group consisting of furniture polishes, floor waxes and shoe creams, or
a body care composition, which is selected in particular from the group consisting of shower gels and shampoos, shaving soaps, shaving foams, bath oils, cosmetic emulsions of the oil-in-water type, of the water-in-oil type and of the water-in-oil-in-water type, such as e.g. skin creams and lotions, face creams and lotions, sunscreen creams and lotions, aftersun creams and lotions, hand creams and lotions, foot creams and lotions, hair removal creams and lotions, aftershave creams and lotions, tanning creams and lotions, hair care products such as e.g. hairsprays, hair gels, setting hair lotions, hair conditioners, permanent and semipermanent hair colorants, hair shaping compositions such as cold waves and hair smoothing compositions, hair tonics, hair creams and hair lotions, deodorants and antiperspirants such as e.g. underarm sprays, roll-ons, deodorant sticks, deodorant creams, products of decorative cosmetics.

The customary ingredients with which fragrances used according to the invention, or inventive fragrance compositions, may be combined, are generally known and described for example in PCT/EP2015/072544, the teaching of which is hereby expressly incorporated by reference.

### EXAMPLES

The examples below are intended to illustrate the invention in more detail. The percentages in the examples are weight percentages unless otherwise indicated.

### Determining the Mean Particle Diameter in Aqueous Suspension/Emulsion Using Light Scattering:

The particle diameter of the w/o/w emulsion or the particle suspension is determined with a Malvern Mastersizer 2000 from Malvern Instruments, England, sample dispersion unit Hydro 2000S according to a standard measurement method which is documented in the literature. The value D[4,3] is the volume-weighted average.

### Determining the Mean Particle Diameter of the Solid:

The microparticles are determined as powder with a Malvern Mastersizer 2000 from Malvern Instruments, England, including powder feed unit Scirocco 2000 according to a standard measurement method which is documented in the literature. The value D[4,3] is the volume-weighted average.

### Determining the Pore Diameter:

The pore diameters were determined by means of scanning electron microscopy (Phenom Pro X). For this purpose, various close-up images were taken and these were retrospectively automatically measured using the ProSuite (FibreMetric) software from Phenom. The pores of a selected region of a particle were identified using the difference in contrast and the surfaces thereof were automatically measured. The diameter for each surface was calculated with the assumption that the surfaces were circular. (Sample size 100 pores).

In the context of the evaluation, only those pores whose pore diameter was at least 20 nm were taken into consideration. Depending on the particle size, the images were recorded, for larger particles with 1600- to 2400-times magnification, and for smaller particles with up to 8000-times magnification.

In order to determine the size of at least 10 pores, only those microparticles whose particle diameter does not deviate from the mean particle diameter of the composition of microparticles by more than 20% were taken into consideration.

The following assumption was made for evaluation of the number of pores based on the total surface area of the microparticle: Since these are spherical particles, the image only shows half the surface of the particle. If the image of a microparticle shows at least 5 pores whose diameter is at least 20 nm and whose diameter is in the range from 1/5000 to 1/5 of the mean particle diameter, then the total surface comprises at least 10 pores.

The evaluation was carried out according to the following procedure:

1. The mean particle diameter D[4,3] of the microparticles was already determined in the microparticle dispersion, using light scattering. The upper and lower limits of the particle diameter of the microparticles which are taken into consideration for determining the pores (±20%) can be calculated from this.
2. The microparticle dispersion was dried.
3. From a sample, in each case 20 images showing multiple microparticles were taken by means of scanning electron microscopy.
4. 20 microparticles were selected whose particle diameter is in the range±20% of the mean particle diameter of the microparticles. The particle diameter thereof was thus measured with the ProSuite (FibreMetric) software from Phenom.
5. The pores of each of these 20 microparticles were measured. For this purpose, the surface areas of the visible pores were measured automatically and the diameter thereof was calculated.
6. The individual values of the pore diameters were checked as to whether their diameter met the condition of being in the range from 1/5000 to 1/5 of the mean particle diameter and being at least 20 nm.
7. The number of pores meeting this condition was determined and multiplied by two.
8. It was verified whether at least 16 microparticles each had on average at least 10 pores.

### Determining the Bulk Density:

The bulk density was determined as specified in DIN-EN ISO 60: 1999.

### Determining the Water Content of the Microparticle Composition

Karl Fischer titration (DIN 51777): For this, approx. 2 g of powder were precisely weighed in and titrated with a 799 GPT titrino by the Karl Fischer method.

### Example 1: Procedure for Preparing the Fillable Spherical Microparticles

Pore former solution: 0.54 g of ammonium carbonate were dissolved in 53.46 g of water (pore former).

Solution of the Aliphatic-Aromatic Polyester and of the Additional Polymer: 15.12 g of PBSeT and 6.48 g of poly(3-hydroxybutyrate-co-3-hydroxyhexanoate) (P(3HB-co-3HHx)) were stirred into 270.0 g of dichlormethane and dissolved with stirring at 25° C.

In order to prepare the w/o emulsion, 54.0 g of pore former solution were emulsified in the solution of the aliphatic-aromatic polyester and the additional polymer for 1 minute at 5 000 rpm with a rotor-stator.

The w/o emulsion thus created was transferred into 419 g of a 0.8% by weight polyvinyl alcohol solution (having a degree of hydrolysis of 88 mol % and a viscosity of 25 mPa*s and proportion of carboxyl groups of 3 mol %) and likewise emulsified with shear and energy input (one minute at 300 rpm with an anchor stirrer).

The w/o/w emulsion produced in this way was subsequently further stirred at 150 rpm with an anchor stirrer, heated slowly to 40° C. while being stirred, and kept at this temperature for 4 hours with a nitrogen flow of 100 l/hour. Thereafter, the microparticle suspension was cooled to room temperature and filtered.

The mean particle diameter after filtration was 257 μm. Water content: <0.5%

### Examples 2 to 3

The procedure was analogous to example 1 with the difference that the polymer mixtures specified in Table 1 composed of aliphatic-aromatic polyester and a copolyester of 3-hydroxybutyrate and 3-hydroxyhexanoate [P(3HB-co-3HHx)] were used for the preparation of the fillable spherical microparticles.

### Example 4

Pore former solution: 0.0225 g of ammonium bicarbonate were dissolved in 4.4775 g of water (pore former).

### Solution of the Aliphatic-Aromatic Polyester and of the Additional Polymer:

1.26 g of PBSeT and 0.54 g of poly(3-hydroxybutyrate-co-3-hydroxyhexanoate) (P(3HB-co-3HHx)) were stirred into 22.5 g of dichloromethane and dissolved with stirring at 25° C. In order to prepare the w/o emulsion, 4.5 g of pore former solution were emulsified in the solution of the aliphatic-aromatic polyester and the additional polymer for 1 minute at 10 000 rpm with a rotor-stator.

The resultant w/o emulsion was transferred into 86 g of a 1% by weight polyvinyl alcohol solution (having a degree of hydrolysis of 88 mol % and a viscosity of 25 mPa*s and proportion of carboxyl groups of 3 mol %) and likewise emulsified with shear and energy input (one minute at 8 000 rpm with a rotor-stator).

The w/o/w emulsion produced in this way was subsequently further stirred at 400 rpm with an anchor stirrer and kept at room temperature for 10 hours with a nitrogen flow of 100 l/hour.

TABLE 1
Fillable spherical microparticles using various polymers

| | | | | Mean |
|---|---|---|---|---|
| | | | | particle |
| | | Concentration | | diameter |
| | Pore | of pore former | | D[4,3] |
| Ex. | former | [% by wt.] | Polymer [wt %] | [μm]¹⁾ |
| 1 | Ammonium | 1.0 | Mixture: 70% PBSeT + | 257 |
| | carbonate | | 30% P(3HB-co-3HHx)^{a} | |
| 2 | Ammonium | 1.0 | Mixture: 70% PBSeT + | 378 |
| | carbonate | | 30% P(3HB-co-3HHx)^{b} | |
| 3 | Ammonium | 1.0 | Mixture: 30% PBSeT + | 195 |
| | carbonate | | 70% P(3HB-co-3HHx)^{b} | |
| 4 | Ammonium | 1.0 | Mixture: 30% PBSeT + | 96 |
| | carbonate | | 70% P(3HB-co-3HHx)^{a} | |

^{a}P(3HB-co-3HHx) comprises 7 mol % 3HH; product Aonilex X131A, commercially available from Kaneka;//^{b}P(3HB-co-3HHx) comprises 11 mol % 3HHx, product Aonilex X151A, commercially available from Kaneka//PBSeT: polybutylene sebacate terephthalate = polyester of 1,4-butanediol and a mixture of sebacic acid and terephthalic acid; product Ecoflex ™ FS Blend A1300 from BASF SE.
¹⁾Determining the particle diameter of the microparticle in the aqueous suspension.

### Abbreviations Used

3HHx=3-hydroxyhexanoate; 3HB=3-hydroxybutyrate; P(3HB-co-3HHx)=copolyester of 3-hydroxybutyric acid and 3-hydroxyhexanoic acid

### Example 5: Procedure for Preparing the Fillable Spherical Microparticles

Pore former solution: 0.54 g of ammonium carbonate were dissolved in 53.46 g of water (pore former).

Solution of the aliphatic-aromatic polyester and of the additional polymer: 15.12 g of PBSeT and 6.48 g of polycaprolactone were stirred into 270.0 g of dichloromethane and dissolved at 25° C. while stirring.

In order to prepare the w/o emulsion, 54.0 g of pore former solution were emulsified in the solution of the aliphatic-aromatic polyester and the additional polymer for 1 minute at 5 000 rpm with a rotor-stator.

The w/o emulsion thus created was transferred into 419 g of a 0.8% by weight polyvinyl alcohol solution (having a degree of hydrolysis of 88 mol % and a viscosity of 25 mPa*s and proportion of carboxyl groups of 3 mol %) and likewise emulsified with shear and energy input (one minute at 300 rpm with an anchor stirrer).

The w/o/w emulsion produced in this way was subsequently further stirred at 150 rpm with an anchor stirrer, heated slowly to 40° C. while being stirred, and kept at this temperature for 4 hours with a nitrogen flow of 100 l/hour. Thereafter, the microparticle suspension was cooled to room temperature and filtered.

The mean particle diameter after filtration was 289 μm: water content <0.5%

### Example 6

The procedure was analogous to example 5 with the difference that the polymer mixtures specified in Table 2 (composed of aliphatic-aromatic polyester and a polycaprolactone) were used for the preparation of the fillable spherical microparticles.

### Example 7: Procedure for Preparing the Fillable Spherical Microparticles

The matrix-forming polymer used was a polymer blend of 70% by weight PBSeT and 30% by weight polycaprolactone. The procedure was as follows:

Pore former solution: 0.54 kg of ammonium carbonate was dissolved in 53.5 kg of water (pore former). Solution of the aliphatic-aromatic polyester: 15.1 kg of PBSeT (as in Example 1) and 6.5 kg of polycaprolactone (as in Example 5) were stirred into 270.0 kg of dichloromethane and dissolved at 25° C. while stirring.

The w/o emulsion was produced by emulsifying the pore former solution in the solution of the aliphatic-aromatic polyester at 170 rpm with a twin-level cross-beam stirrer for 15 minutes.

The resulting w/o emulsion was transferred into 423 kg of a 0.8% by weight aqueous polyvinyl alcohol solution and likewise emulsified with shear and energy input (one minute at 120 rpm using a round anchor stirrer).

Stirring of the w/o/w emulsion thus created with an impeller stirrer was then continued at 120 rpm, while reducing the pressure to 800 mbar and gradually increasing the jacket temperature to 40° C. and keeping it at this temperature for 4 hours. Thereafter, the microparticle suspension was cooled to room temperature, filtered and dried at 37° C.

The average particle diameter D[4,3] determined from the aqueous suspension was 110 μm.

TABLE 2
Fillable spherical microparticles using various polymers

| | | | | Mean |
|---|---|---|---|---|
| | | | | particle |
| | | Concentration | | diameter |
| | Pore | of pore former | | D[4,3] |
| Ex. | former | [% by wt.] | Polymer [wt %] | [μm]¹⁾ |
| 5 | Ammonium | 1.0 | Mixture: 70% PBSeT + | 289 |
| | carbonate | | 30% polycaprolactone | |
| 6 | Ammonium | 1.0 | Mixture: 30% PBSeT + | 277 |
| | carbonate | | 70% polycaprolactone | |
| 7 | Ammonium | 1.0 | Mixture: 70% PBSeT + | 110 |
| | carbonate | | 30% polycaprolactone | |

PBSeT: polybutylene sebacate terephthalate as in Example 1
Polycaprolactone: commercially available from Perstorp under the trade name Capa ™ 6506.
Polycaprolactone having an approximate Mw of 50 000 and a melting point of 58-60° C.

TABLE 3
Detailled characterization of spherical microparticles
using various polymer mixtures.

| | | | | Calculated upper | | |
|---|---|---|---|---|---|---|
| | | Smallest | | and lower limits | | |
| | | and largest | | of the pore | | |
| | | pore diameter | | diameter [μm] | | |
| | Mean particle | measured [μm] | | Lower | Upper | Number of |
| Ex. | diameter [μm] | Min | Max | limit¹⁾ | limit²⁾ | pores ≥10 |
| 1 | 257 | 1.9 | 11.7 | 0.05 | 51.4 | Met |
| 2 | 378 | 2.1 | 13.0 | 0.08 | 75.6 | Met |
| 3 | 195 | 1.3 | 4.8 | 0.04 | 39 | Met |
| 4 | 96 | 0.4 | 3.4 | 0.02 | 19.2 | Met |
| 5 | 289 | 1.9 | 7.4 | 0.06 | 57.8 | Met |
| 6 | 277 | 2.5 | 11.2 | 0.06 | 55.4 | Met |

¹⁾ 1/5000 of the mean particle diameter of the microparticles
²⁾⅕ of the mean particle diameter of the microparticles

### Examples 8a to 8c: Impregnation of the Spherical Microparticles by Spray Application

500 g of the microparticles from Example 7 were initially charged in a ploughshare mixer and sprayed with 1000 g of a solution A at 20° C. by means of a one-phase nozzle having a nozzle diameter of 0.5 mm (spray pressure 2 bar) over 2 min (flow rate 500 ml/min).

### Example 8a): Solution a Used was a 10% by Weight Solution of L-Menthol in 1,2-Propylene Glycol

L-Menthol with a purity of >99.7% is commercially available under the trade name L-Menthol FCC from BASF SE.

### Example 8b): Solution a Used was a 10% by Weight Solution of Rose Oxide 90 in 1,2-Propylene Glycol

Rose oxide 90 (chemical name: tetrahydro-4-methyl-2-(2-methylprop-1-enyl)pyran)) with a purity (sum of isomers, CGC) 98.0% (area), cis-isomer 90.0-95.0% (CGC, area)/trans-isomer 5-10% (CGC, area) is commercially available from BASF SE.

### Example 8c): Solution a Used was a 10% by Weight Solution of Dihydrorosan in 1,2-Propylene Glycol

Dihydrorosan (chemical name tetrahydro-2-isobutyl-4-methyl-2H-pyran) with a purity (sum of isomers, GC)≥98.0% (area), having a proportion of cis-isomer of 65-85% (area) and trans-isomer of 15-35% (area), commercially available from BASF SE.

1.-16. (canceled)

17. A composition of spherical microparticles composed of a wall material and at least one cavity that comprises a gas and/or a liquid, which have pores on the surface thereof, wherein the spherical microparticles have a mean particle diameter of 10-600 μm and wherein at least 80% of those microparticles, the particle diameter of which does not deviate from the mean particle diameter of the microparticles of the composition by more than 20%, each have on average at least 10 pores, the diameter of which is in the range from 1/5000 to 1/5 of the mean particle diameter, and, furthermore, the diameter of each of these pores is at least 20 nm, wherein the wall material consists of a composition comprising at least one aliphatic-aromatic polyester and at least one additional polymer, wherein the additional polymer is selected from the group consisting of polyhydroxy fatty acids, poly(p-dioxanones), polyanhydrides, polyesteramides, polysaccharides and proteins.

18. The composition of spherical microparticles according to claim 17, wherein the aliphatic-aromatic polyester is an ester of an aliphatic dihydroxy compound esterified with a composition of aromatic dicarboxylic acid and aliphatic dicarboxylic acid.

19. The composition of spherical microparticles according to claim 17, wherein the aliphatic-aromatic polyester is selected from polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST).

20. The composition of spherical microparticles according to claim 17, wherein the composition forming the wall material comprises at least one polymer having a glass transition temperature or a melting point in the range from 45 to 140° C.

21. The composition of spherical microparticles according to claim 17, wherein the wall material has a solubility in dichloromethane of at least 50 g/l at 25° C.

22. The composition of spherical microparticles according to claim 17, wherein the additional polymer is at least one polyhydroxy fatty acid.

23. A method for preparing a composition of spherical microparticles, comprising
a) preparing an emulsion from water or an aqueous solution of a pore former as discontinuous phase and a continuous phase comprising a solution of at least one aliphatic-aromatic polyester and at least one additional polymer selected from the group consisting of polyhydroxy fatty acids, poly(p-dioxanones), polyanhydrides, polyesteramides, polysaccharides and proteins, in a water-immiscible solvent,
b) emulsifying the emulsion obtained in a) in water in the presence of at least one dispersant to give a w/o/w emulsion having droplets with a mean size of 1-600 μm, and removing the water-immiscible solvent at a temperature in the range from 20 to 80° C.,
c) separating off the spherical microparticles formed in method step b) and optionally drying the spherical microparticles.

24. A composition of spherical microparticles obtained by the method according to claim 23.

25. A carrier substance for filling with at least one aroma chemical comprising the composition of spherical microparticles according to claim 17.

26. The method according to claim 23, further comprising impregnating the optionally dried spherical microparticles with at least one aroma chemical.

27. A method for preparing an aroma chemical preparation, comprising impregnating the composition of spherical microparticles according to claim 17 with at least one aroma chemical.

28. The method for preparing an aroma chemical preparation according to claim 27, wherein the spherical microparticles are suspended in a liquid aroma chemical or in a solution of at least one aroma chemical.

29. An aroma chemical preparation obtained by the method of claim 26.

30. A composition comprising the aroma chemical preparation according to claim 29, wherein the composition is selected from perfumes, washing and cleaning compositions, cosmetic compositions, body care compositions, hygiene articles, food, food supplements, scent dispensers or fragrances.

31. A composition comprising the composition of spherical microparticles according to claim 17, in a proportion by weight of 0.01 to 99.9% by weight, based on the total weight of the composition.

32. A method for the controlled release of aroma chemicals comprising incorporating the aroma chemical preparation according to claim 29 into a perfume, washing and cleaning composition, cosmetic composition, body care composition, hygiene article, food, food supplement, scent dispenser or fragrance.

33. The composition of spherical microparticles according to claim 17, wherein the additional polymer is at least one polycaprolactone
